SEQUENCE LISTING (4 sequences)

SEQ ID NO: 1 - protein, 16 aa, synthetic construct
RADARADARADARADA

SEQ ID NO: 2 - protein, 22 aa, synthetic construct
RADARADARADARADAGGRGDS

SEQ ID NO: 3 - protein, 30 aa, synthetic construct
RADARADARADARADAGGFPGERGVEGPGP

SEQ ID NO: 4 - protein, 13 aa, synthetic construct
GFFYGGGWRESAI

(19) United States
(12) Patent Application Publication
(10) Pub. No.: US 2025/0041208 A1
(43) Pub. Date: Feb. 6, 2025

(54) NEW INJECTABLE COMBINATION FORMULATION

(71) Applicant: NANEXA AB, Uppsala (SE)

(72) Inventors: Anders JOHANSSON, Uppsala (SE); Mårten ROOTH, Uppsala (SE); Joel HELLRUP, Uppsala (SE); David WESTBERG, Uppsala (SE)

(21) Appl. No.: 18/717,160

(22) PCT Filed: Dec. 8, 2022
(86) PCT No.: PCT/GB2022/053128

§ 371 Date: Jun. 6, 2024

(30) Foreign Application Priority Data
Dec. 8, 2021 (GB) 2117703.5
Jun. 10, 2022 (GB) 2208520.3
Sep. 30, 2022 (GB) 2214380.4

(51) Int. Cl. A61K 9/00; A61K 9/50; A61K 31/192; A61K 31/196; A61K 31/405; A61K 31/407; A61K 31/4439; A61K 31/454; A61K 31/4545; A61K 31/5415; A61K 31/569; A61K 31/635; A61K 31/706

(52) U.S. Cl. CPC A61K 9/0024; A61K 9/501; A61K 31/192; A61K 31/196; A61K 31/405; A61K 31/407; A61K 31/4439; A61K 31/454; A61K 31/4545; A61K 31/5415; A61K 31/569; A61K 31/635; A61K 31/706

(57) ABSTRACT

There is provided a pharmaceutical or veterinary formulation comprising: (a) a biologically-active agent in admixture with a pharmaceutically- or veterinarily-acceptable extended-release component; (b) an antiinflammatory agent; and (c) a pharmaceutically- or veterinarily-acceptable injectable carrier. An extended-release component may also be applied to the antiinflammatory agent. The formulation may provide for the delayed or sustained release of biologically active ingredient without producing an inflammatory response after injection, and s preferably provided in the form of: (1) a plurality of particles having a weight-, number-, or volume-based mean diameter that is between about 10 nm and about 700 μm, which particles comprise solid corms comprising a biologically-active agent coated with a coating comprising at least one coating material applied by way of a gas phase deposition technique; (2) which particles are suspended in a carrier system comprising a pharmaceutically-acceptable or veterinarily-acceptable vehicle; and (3) which formulation further includes an antiinflammatory agent, which is optionally in the form of particles that are coated with a coating comprising at least one coating material applied by way of a gas phase deposition technique. Said coated particles are preferably synthesized via atomic layer deposition.

### FIELD OF THE INVENTION

This Invention relates to a new formulation for use in for example the field of drug delivery.

### PRIOR ART AND BACKGROUND

The listing or discussion of an apparently prior-published document in this specification should not necessarily be taken as an acknowledgement that the document is part of the state of the art or common general knowledge.

In the field of drug delivery, the ability to control the profile of drug release is of critical importance. It is desirable to ensure that active ingredients are released at a desired and predictable rate in vivo following administration, in order to ensure the optimal pharmacokinetic profile.

In the case of any sustained release composition, it is of critical importance that its release profile shows minimal initial rapid release of active ingredient, that is a large concentration of drug in plasma shortly after administration. Such a ‘burst’ release will result in unwanted, high concentrations active ingredient, and may be hazardous in the case of drugs that have a narrow therapeutic window or drugs that are toxic at high plasma concentrations, such as cytotoxic drugs.

In the case of an injectable suspension of an active ingredient, it is also important that the size of the suspended particles is controlled so that they can be injected through a needle. If large, aggregated particles are present, they will not only block the needle, through which the suspension is to be injected, but also will not form a stable suspension within (i.e. they will instead tend to sink to the bottom of) the injection liquid.

There is thus a general need in the art for effective and/or improved drug transport and delivery systems.

Atomic layer deposition (ALD) is a technique that is employed to deposit thin films comprising a variety of materials, including organic, biological, polymeric and, especially, inorganic materials, such as metal oxides, on solid substrates. It is an enabling technique for atomic and close-to-atomic scale manufacturing (ACSM) of materials, structures, devices and systems in versatile applications (see, for example, Zhang et al. Nanomanuf. Metrol. 2022, https://doi.org/10.1007/s41871-022-00136-8). Based on its self-limiting characteristics, ALD can achieve atomic-level thickness that is only controlled by adjusting the number of growth cycles. Moreover, multilayers can be deposited, and the properties of each layer can be customized at the atomic level.

Due to its atomic-level control, ALD is used as a key technique for the manufacturing of, for example, next-generation semiconductors, or in atomic-level synthesis of advanced catalysts as well as in the precise fabrication of nanostructures, nanoclusters, and single atoms (see, for example, Zhang et al. vide supra).

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor.

This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and conducting a re-dispersion/agitation step to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in water and sonicating, which resulted in deagglomeration, and the breaking up of contact points between individual particles of coated active substance.

The particles were then loaded back into the reactor and the steps of ALD coating of the powder, and deagglomerating the powder were repeated 3 times, to a total of 4 series of cycles. This process has been found to allow for the formation of coated particles that are, to a large extent, free of pinholes (see also, Hellrup et al., Int. J. Pharm., 529, 116 (2017)).

As described hereinafter, when testing a novel, injectable pharmaceutical, in which ALD is used to coat microparticles of a drug with metal oxide coating layers, which coated particles are then suspended in an aqueous vehicle, in human patients, an unexpected inflammatory response was observed. This problem may be reduced by employing an antiinflammatory agent in conjunction with ALD-coated suspensions of APIs for injection.

### DISCLOSURE OF THE INVENTION

According to a first aspect of the invention, there is provided an injectable pharmaceutical or veterinary formulation comprising:
(a) a biologically-active agent in admixture with a pharmaceutically- or veterinarily-acceptable extended-release component;
(b) an antiinflammatory agent; and
(c) a pharmaceutically- or veterinarily-acceptable injectable carrier, which formulations are hereinafter referred to as ‘the formulations of the invention’.

By ‘pharmaceutically- or veterinarily-acceptable extended-release component’, we include components that provide for the formation of a so-called ‘depot’ or ‘depot composition’ after (e.g. intratumoral or, more preferably, subcutaneous or intramuscular) Injection, and thus a controlled-release, sustained release and long-acting or prolonged release of active ingredient(s). Such components include aqueous (or water-miscible) components (e.g. an aqueous solution of gelatin or polyvinylpyrrolidone), oleaginous-based (or water-Immiscible) components, polymer-based microsphere components or polymer-based in-situ gel forming components.

Hydrophilic polymers may form a gel in situ in the local environment driven by different mechanisms, e.g. either by temperature (such as poly(D,L-lactide-co-glycolide) (PLGA)/polyethylene glycol (PEG) triblock copolymers (PLGA-PEG-PLGA), polyethylene glycol-poly(L-alanine) (PEG-PLA), poly(N-isopropyl acrylamide), or soluble extracellular matrix (ECM)/methylcellulose), by pH (such as PEG-diacrylate (PEGDA), acrylic acid or alginate), or by ionic concentration (such as alginate/multi walled carbon nanotubes, alginate/PEG/hyaluronic acid, or alginate/PEG). Hydrophilic polymers may alternatively form a gel in situ by, for example, self-assembly, such as peptides, e.g. RADA16 peptides (RADARADARADARADA) with a fibronectin attachment motif (RADA16-GG-RGDS) or a collagen type-1 derived motif (RADA16-GGFPGERGVEGPGP), fluorenylmethoxycarbonyl (Fmoc) dipeptides, Nap-GFFYGGGWRESAI/TIP-1 crosslinker, or Leucine-α/β-dehydrophenylalanine. Hydrophilic polymers may alternatively form a gel in situ by, for example, covalent bonding driven by different mechanisms, e.g. either by photo-initiation (such as gelatin-methacrylate, or gelatin-methacrylate/hyaluronic acid (HA)-methacrylate), or by reactive precursors (such as 8-Arm PEG cysteine/N-hydroxysuccinimide, carboxymethyl chitosan/dextran, or konjac glucomannan-tyramine/heparin-tyramine).

Hydrophilic in situ gel forming polymers may be derived from, for example, natural or synthetic sources. Examples of naturally occurring polymers include polysaccharides, such as chitosan, alginate, hyaluronic acid (HA), dextran, starch, or proteins, such as albumin, collagen or gelatin. Examples of synthetic polymers for nanoparticle formulations include polyesters, polyanhydrides or poly-alkyl-cyanoacrylates, such as poly(ethylene glycol) (PEG), polyacrylamide (PAM), poly(N-isopropyl acrylamide) (PNIPAAM), poly(vinyl alcohol) (PVA), poly(vinyl ether) (PVE), poly(N,Ndiethylacrylamide) (PDEAM), poly(N-vinyl caprolactam) (PNVCa), poly(methyl methacrylate) (PMMA), or poly(oligo(ethylene glycol) methyl ether metacrylate) (PoEGMA). More complex, hydrogel systems may be used, such as either co-polymers, e.g. poly(D,L-lactide-co-glycolide) (PLGA)/PEG triblock copolymers (PLGA-PEG-PLGA), where multiple backbone groups may be crosslinked together, or Inter-penetrating networks (IPNs), where a polymer mesh may be constructed from the binding of oligomer chains within an already assembled polymeric scaffold.

The polymers may be combined with other materials, such as degradable polymer coatings on titanium oxide (titania) nanotubes, to achieve a controlled drug release, e.g. as orthopedic drug-eluting implants.

Hydrating Ionic salts, e.g. calcium phosphate or calcium sulfate, may be injected as a suspension during hydration and used for medical drug depot applications as they are considered to be both biocompatible and biodegradable.

The extended-release component may be applied to the aforementioned biologically-active agent alone or may also be applied to the antiinflammatory agent within a formulation of the invention. If extended-release components are applied to both of the active agents, these extended-release components may be the same or they may be different in terms of their composition and/or function.

Whatever extended-release components are applied to the respective active agents, following intratumoral or, preferably, subcutaneous or intramuscular injection of a formulation of the invention to a subject, at least one depot composition is formed that provides for an extended release of at least said biologically active agent.

When extended-release components are applied to both biologically active agent and antiinflammatory agent, it is preferred that, following intratumoral or, more preferably, subcutaneous or intramuscular injection of a formulation of the invention to a subject, a depot is formed that provides for an extended release of said biologically active agent and said antiinflammatory agent over time, at essentially the same rate, and/or over essentially the same period of time.

In other words, it is preferred that the biologically active agent and the antiinflammatory agent both form depots (or together form a single depot composition) after administration, meaning that they have concomitant release profiles. That is, release of both the biologically active agent and the antiinflammatory agent is uniform and/or constant over an extended (and/or essentially the same) period of time.

By ‘essentially the same rate of release of the biologically active agent and the antiinflammatory agent and/or over essentially the same period of time’, we include not only that the amount of active ingredients that are released from the, or their respective, extended release depot composition(s) per unit time (e.g. μg/hour or μg/day) is essentially the same, but also (in addition to and/or instead of this) that overall release may take place over essentially the same extended time period, following injection. By ‘essentially the same’ in this context, we include that relevant changes in the concentrations of the respective active ingredients in plasma (as may be measured using techniques that are routine and/or are standard to those skilled in the art at specific time points after injection) are within about ±50% of each other, including ±30%, such as ±20% of each other.

It is believed that the feature of extended release of the biologically active agent and the antiinflammatory agent over time, at essentially the same rate, and over essentially the same period of time may allow the administration of biologically active agents that may, or are expected to, give rise to localized inflammation when injected into, and exposed to, e.g. tumoral, more especially muscular and/or subcutaneous tissue. Subcutaneous or intramuscular injections are often used for extended release, however, one significant limitation to such routes of administration is that such routes are often limited to non-irritant biologically active agents (see, for example, Muralidhar et al, Asian Journal of Biomaterial Research, 3, 6 (2017)). It is believed that the present invention may allow the use of biologically active agents that are, may be, and/or are expected to be, irritant, in that they may give rise to inflammation at e.g. a local level.

Furthermore, inflammation may provide both key mutations and the proper environment to foster tumor growth and, consequently, may play role in the establishment, progression, and/or aggressiveness of various malignancies. Many anti-inflammatory agents can alter the tumors themselves or the tumor microenvironment, potentially decreasing migration, increasing apoptosis, and increasing sensitivity to other therapies (see, for example, Rayburn et al, Molecular and Cell Pharmacology, 1(1), 29 (2009)). It is believed that the present invention may allow the use of biologically active agents that are, may be, and/or are expected to give rise to localized inflammation when injected into, and exposed to, for example, tumoral tissues.

Thus, in an embodiment of the invention, there is provided an injectable pharmaceutical or veterinary formulation comprising:
(a) a biologically-active agent that gives, may give, or is expected to give, rise to localized inflammation when injected into, and exposed to tumoral, for example muscular and/or subcutaneous tissue;
(b) an antiinflammatory agent; and
(c) one or more extended-release components that, following intratumoral or, more preferably subcutaneous or intramuscular injection of the formulation to a subject, forms a depot composition that provides for an extended release of said biologically active agent and, optionally, said antiinflammatory agent, over time.

In accordance with this aspect of the invention, it is preferred that the formulation does not include an anaesthetic and/or analgesic agent.

Further, in accordance with this latter aspect of the invention, when the one or more extended-release components provide for extended release of both said biologically active agent and said antiinflammatory agent, such release is preferably at essentially the same rate, and over essentially the same period of time such that the antiinflammatory agent reduces the degree of inflammation resulting from said biologically active agent during its release into said tissue (as hereinbefore described).

By ‘agent that gives, may give, or is expected to give, rise to a localized inflammation’ we include those agents (including those described hereinafter) that are so known and/or expected, based on information received following experiments (e.g. as described hereinafter), or from elsewhere (e.g. from the literature or product labels), to give rise to such localized inflammation.

In another embodiment, there is provided an injectable pharmaceutical or veterinary formulation comprising:
(a) an injectable anticancer drug (e.g. as defined hereinafter, such as azacitidine or lenalidomide);
(b) an antiinflammatory agent; and
(c) an extended-release component that, following intratumoral, or more preferably, subcutaneous or intramuscular injection of said formulation to a subject, forms a depot composition that provides for an extended release of said anticancer drug and (optionally) said an antiinflammatory agent, over time.

In the context of the present invention, the terms ‘depot composition’, ‘depot-forming composition’ and ‘depot formulation’ are used interchangeably when referring to a composition which releases slowly over time to permit less frequent administration of a medication.

It is preferred that the extended-release of the biologically-active agent following injection is obtained by encapsulating small, injectable (e.g. micro) particles comprising said biologically-active agent with at least one coating material applied by way of a gas phase deposition technique.

In a preferred aspect of the invention, there is thus provided a pharmaceutical or veterinary formulation comprising:
(a) a plurality of particles having a weight-, number-, or volume-based mean diameter that is between about 10 nm and about 700 μm, which particles comprise solid cores comprising a biologically-active agent coated with a coating comprising at least one coating material applied by way of a gas phase deposition technique;
(b) which particles are suspended in a carrier system comprising a pharmaceutically-acceptable or veterinarily-acceptable vehicle; and
(c) which formulation further includes an antiinflammatory agent, which formulations are also hereinafter referred to as ‘the formulations of the invention’.

In accordance with this aspect of the invention, it is preferred that at least the biologically-active agent is coated as described above. The antiinflammatory agent that is also included within this aspect of the invention may or may not be presented in conjunction with an extended-release component as described herein. If so presented, such an extended-release component may or may not be in the form of a similar coating on cores comprising said antiinflammatory agent, which coating comprises at least one coating material applied by way of a gas phase deposition technique.

The term ‘solid’ will be well understood by those skilled in the art to include any form of matter that retains its shape and density when not confined, and/or in which molecules are generally compressed as tightly as the repulsive forces among them will allow. The solid cores in accordance with this aspect of the invention have at least a solid exterior surface onto which a layer of coating material can be deposited. The interior of the solid cores may be also solid or may instead be hollow. For example, if the particles are spray dried before they are placed into the reactor vessel, they may be hollow due to the spray drying technique.

Formulations of the invention comprise a pharmacologically-effective amount of said biologically-active agent. Preferably, the solid cores of this aspect of the formulation of the invention comprise said pharmacologically-effective amount of biologically-active agent.

Such solid cores may consist essentially of, or may comprise, biologically-active agent (which agent may hereinafter be referred to interchangeably as a ‘drug’, and ‘active pharmaceutical ingredient (API)’ and/or an ‘active Ingredient’). The term ‘biologically-active agent’ also includes biopharmaceuticals and/or biologics. Biologically-active agents can also comprise a mixture of two or more different APIs, either as different API particles or as particles comprising more than one API.

By ‘consists essentially’ of biologically-active agent(s), we include that the aforementioned solid core is essentially comprised only of biologically-active agent(s), i.e. It is free from non-biologically active substances, such as excipients, carriers and the like (vide infra). This means that the core may comprise less than about 5%, such as less than about 3%, including less than about 2%, e.g. less than about 1% of such other excipients and/or other active substances.

In the alternative, cores comprising biologically-active agent may include such an agent in admixture with one or more pharmaceutical ingredients, such as one or more pharmaceutically-acceptable excipients, such as adjuvants, diluents or carriers, and/or may include other biologically-active ingredients, including one or more of the essential antiinflammatory agents that are included in a formulation of the invention. Biologically-active agents may thus be presented in combination (e.g. In admixture or as a complex) with another active substance, such as one or more of the essential antiinflammatory agents that are included in a formulation of the invention.

Biologically-active agents may be presented in a crystalline, a part-crystalline and/or an amorphous state. Biologically-active agents may further comprise any substance that is in the solid state, or which may be converted into the solid state, at about room temperature (e.g. about 18° C.) and about atmospheric pressure, irrespective of the physical form. Such agents should also remain in the form of a solid whilst being coated in the gas phase deposition (e.g. ALD) reactor and also should not decompose physically or chemically to an appreciable degree (i.e. no more than about 10% w/w) whilst being coated, or after having been covered by at least one of the aforementioned layers of coating materials.

As used herein, the term ‘biologically-active agent’, or similar and/or related expressions, generally refer(s) to any agent, or drug, capable of producing some sort of physiological effect (whether in a therapeutic or prophylactic capacity against a particular disease state or condition) in a living subject, including, in particular, mammalian and especially human subjects (patients).

Biologically-active agents may, for example, be selected from an analgesic, an anaesthetic, an anti-ADHD agent, an anorectic agent, an antiaddictive agent, an antibacterial agent, an antimicrobial agent, an antifungal agent, an antiviral agent, an antiparasitic agent, an antiprotozoal agent, an anthelmintic, an ectoparasiticide, a vaccine, an anticancer agent, an antimetabolite, an alkylating agent, an antineoplastic agent, a topoisomerase inhibitor, an immunomodulator, an immunostimulant, an immunosuppressant, an anabolic steroid, an anticoagulant agent, an antiplatelet agent, an anticonvulsant agent, an antidementia agent, an antidepressant agent, an antidote, an antihyperlipidemic agent, an antigout agent, an antimalarial, an antimigraine agent, an antiparkinson agent, an antipruritic agent, an antipsoriatic agent, an antiemetic, an anti-obesity agent, an antiasthma agent, an antibiotic, an antidiabetic agent, an antiepileptic, an antifibrinolytic agent, an antihemorrhagic agent, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, an antiparkinsonian agent, a free radical scavenging agent, a growth factor, a fibrate, a bile acid sequestrants, a cicatrizant, a glucocorticoid, a mineralocorticoid, a haemostatic, a hallucinogen, a hypothalamic-pituitary hormone, an immunological agent, a laxative agent, a antidiarrhoeals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthalmological, an ontological, a H1 antagonist, a H2 antagonist, a proton pump inhibitor, a prostaglandin, a radiopharmaceutical, a hormone, a sedative, an anti-allergic agent, an appetite stimulant, a steroid, a sympathomimetic, a thrombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a radiopharmaceutical, a senotherapeutic, a vitamin, or a mixture of any of these.

The biologically-active agent may also be a cytokine, a peptidomimetic, a peptide, a protein, a toxoid, a serum, an antibody, a vaccine, a nucleoside, a nucleotide, a portion of genetic material, a nucleic acid, or a mixture thereof. Non-limiting examples of therapeutic peptides/proteins are as follows: lepirudin, cetuximab, dornase alfa, denileukin diftitox, etanercept, bivalirudin, leuprolide, alteplase, interferon alfa-n1, darbepoetin alfa, reteplase, epoetin alfa, salmon calcitonin, interferon alfa-n3, pegfilgrastim, sargramostim, secretin, peginterferon alfa-2b, asparaginase, thyrotropin alfa, antihemophilic factor, anakinra, gramicidin D, intravenous immunoglobulin, anistreplase, insulin (regular), tenecteplase, menotropins, interferon gamma-1b, interferon alfa-2a (recombinant), coagulation factor VIIa, oprelvekin, palifermin, glucagon (recombinant), aldesleukin, botulinum toxin Type B, omalizumab, lutropin alfa, insulin lispro, insulin glargine, collagenase, rasburicase, adalimumab, imiglucerase, abciximab, alpha-1-proteinase inhibitor, pegaspargase, interferon beta-1a, pegademase bovine, human serum albumin, eptifibatide, serum albumin lodinated, infliximab, follitropin beta, vasopressin, interferon beta-1b, hyaluronidase, rituximab, basiliximab, muromonab, digoxin immune Fab (ovine), ibritumomab, daptomycin, tositumomab, pegvisomant, botulinum toxin type A, pancrelipase, streptokinase, alemtuzumab, alglucerase, capromab, laronidase, urofollitropin, efalizumab, serum albumin, choriogonadotropin alfa, antithymocyte globulin, fligrastim, coagulation factor IX, becaplermin, agalsidase beta, interferon alfa-2b, oxytocin, enfuvirtide, palivizumab, daclizumab, bevacizumab, arcitumomab, eculizumab, panitumumab, ranibizumab, idursulfase, alglucosidase alfa, exenatide, mecasermin, pramlintide, galsulfase, abatacept, cosyntropin, corticotropin, insulin aspart, Insulin detemir, insulin glulisine, pegaptanib, nesiritide, thymalfasin, defibrotide, natural alpha interferon/multiferon, glatiramer acetate, preotact, teicoplanin, canakinumab, ipilimumab, sulodexide, tocilizumab, teriparatide, pertuzumab, rilonacept, denosumab, liraglutide, semaglutide, golimumab, belatacept, buserelin, velaglucerase alfa, tesamorelin, brentuximab vedotin, taliglucerase alfa, belimumab, aflibercept, asparaginase Erwinia chrysanthemi, ocriplasmin, glucarpidase, teduglutide, raxibacumab, certolizumab pegol, insulin isophane, epoetin zeta, obinutuzumab, fibrinolysin aka plasmin, follitropin alpha, romiplostim, lucinactant, natalizumab, aliskiren, ragweed pollen extract, secukinumab, somatotropin (recombinant), drotrecogin alfa, alefacept, OspA lipoprotein, urokinase, abarelix, sermorelin, aprotinin, gemtuzumab ozogamicin, satumomab pendetide, albiglutide, antithrombin alfa, antithrombin III (human), asfotase alfa, atezolizumab, autologous cultured chondrocytes, beractant, blinatumomab, C1 esterase inhibitor (human), coagulation factor XIII A-subunit (recombinant), conestat alfa, daratumumab, desirudin, dulaglutide, elosulfase alfa, evolocumab, fibrinogen concentrate (human), filgrastim-sndz, gastric intrinsic factor, hepatitis B immune globulin, human calcitonin, human Clostridium tetani toxoid immune globulin, human rabies virus immune globulin, human Rho(D) immune globulin, human Rho(D) immune globulin, hyaluronidase (human, recombinant), idarucizumab, immune globulin (human), vedolizumab, ustekinumab, turoctocog alfa, tuberculin purified protein derivative, simoctocog alfa, siltuximab, sebelipase alfa, sacrosidase, ramucirumab, prothrombin complex concentrate, poractant alfa, pembrolizumab, peginterferon beta-1a, ofatumumab, obiltoxaximab, nivolumab, necitumumab, metreleptin, methoxy polyethylene glycol-epoetin beta, mepolizumab, ixekizumab, insulin degludec, insulin (porcine), insulin (bovine), thyroglobulin, anthrax immune globulin (human), anti-inhibitor coagulant complex, brodalumab, C1 esterase inhibitor (recombinant), chorionic gonadotropin (human), chorionic gonadotropin (recombinant), coagulation factor X (human), dinutuximab, efmoroctocog alfa, factor IX complex (human), hepatitis A vaccine, human varicella-zoster immune globulin, ibritumomab tiuxetan, lenograstim, pegloticase, protamine sulfate, protein S (human), sipuleucel-T, somatropin (recombinant), susoctocog alfa and thrombomodulin alfa.

Non-limiting examples of drugs which may be used according to the present invention are all-trans retinoic acid (tretinoin), alprazolam, allopurinol, amiodarone, amlodipine, asparaginase, astemizole, atenolol, azathioprine, azelatine, beclomethasone, bendamustine, bleomycin, budesonide, buprenorphine, butalbital, capecitabine, carbamazepine, carbidopa, carboplatin, cefotaxime, cephalexin, chlorambucil, cholestyramine, ciprofloxacin, cisapride, cisplatin, clarithromycin, clonazepam, clozapine, cyclophosphamide, cyclosporin, cytarabine, dacarbazine, dactinomycin, daunorubicin, diazepam, diclofenac sodium, digoxin, dipyridamole, divalproex, dobutamine, docetaxel, doxorubicin, doxazosin, enalapril, epirubicin, erlotinib, estradiol, etodolac, etoposide, everolimus, famotidine, felodipine, fentanyl citrate, fexofenadine, filgrastim, finasteride, fluconazole, flunisolide, fluorouracil, flurbiprofen, fluralaner, fluvoxamine, furosemide, gemcitabine, glipizide, gliburide, ibuprofen, ifosfamide, imatinib, Indomethacin, Irinotecan, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, mercaptopurine, mesna, methotrexate, methylprednisolone, midazolam, mitomycin, mitoxantrone, moxidectine, mometasone, nabumetone, naproxen, nicergoline, nifedipine, norfloxacin, omeprazole, oxaliplatin, paclitaxel, phenyloin, piroxicam, procarbazine, quinapril, ramipril, risperidone, rituximab, sertraline, simvastatin, sulindac, sunitinib, temsirolimus, terbinafine, terfenadine, thioguanine, trastuzumab, triamcinolone, valproic acid, vinblastine, vincristine, vinorelbine, zolpidem, or pharmaceutically-acceptable salts of any of these.

Formulations of the invention may comprise benzodiazipines, such as alprazolam, chlordiazepoxide, clobazam, clorazepate, diazepam, estazolam, flurazepam, lorazepam, oxazepam, quazepam, temazepam, triazolam and pharmaceutically-acceptable salts of any of these.

Anaesthetics that may also be employed in the formulations of the invention may be local or general. Local anaesthetics that may be mentioned include amylocaine, ambucaine, articaine, benzocaine, benzonatate, bupivacaine, butacaine, butanilicaine, chloroprocaine, cinchocaine, cocaine, cyclomethycaine, dibucaine, diperodon, dimethocaine, eucaine, etidocaine, hexylcaine, fomocaine, fotocaine, hydroxyprocaine, isobucaine, levobupivacaine, lidocaine, mepivacaine, meprylcaine, metabutoxycaine, nitracaine, orthocaine, oxetacaine, oxybuprocaine, parethoxycaine, phenacaine, piperocaine, piridocaine, pramocaine, prilocaine, primacaine, procaine, procainamide, proparacaine, propoxycaine, pyrrocaine, quinisocaine, ropivacaine, trimecaine, tolycaine, tropacocaine, or pharmaceutically-acceptable salts of any of these.

Psychiatric drugs may also be employed in the formulations of the invention. Psychiatric drugs that may be mentioned include 5-HTP, acamprosate, agomelatine, alimemazine, amfetamine, dexamfetamine, amisulpride, amitriptyline, amobarbital, amobarbital/secobarbital, amoxapine, amphetamine(s), aripiprazole, asenapine, atomoxetine, baclofen, benperidol, bromperidol, bupropion, buspirone, butobarbital, carbamazepine, chloral hydrate, chlorpromazine, chlorprothixene, citalopram, clomethiazole, clomipramine, clonidine, clozapine, cyclobarbital/diazepam, cyproheptadine, cytisine, desipramine, desvenlafaxine, dexamfetamine, dexmethylphenidate, diphenhydramine, disulfiram, divalproex sodium, doxepin, doxylamine, duloxetine, enanthate, escitalopram, eszopiclone, fluoxetine, flupenthixol, fluphenazine, fluspirilen, fluvoxamine, gabapentin, glutethimide, guanfacine, haloperidol, hydroxyzine, iloperidone, imipramine, lamotrigine, levetiracetam, levomepromazine, levomilnacipran, lisdexamfetamine, lithium salts, lurasidone, melatonin, melperone, meprobamate, metamfetamine, nethadone, methylphenidate, mianserin, mirtazapine, moclobemide, nalmefene, naltrexone, niaprazine, nortriptyline, olanzapine, ondansetron, oxcarbazepine, paliperidone, paroxetine, penfluridol, pentobarbital, perazine, pericyazine, perphenazine, phenelzine, phenobarbital, pimozide, pregabalin, promethazine, prothipendyl, protriptyline, quetlapine, ramelteon, reboxetine, reserpine, risperidone, rubidium chloride, secobarbital, selegiline, sertindole, sertraline, sodium oxybate, sodium valproate, sodium valproate, sulpiride, thioridazine, thiothixene, tianeptine, tizanidine, topiramate, tranylcypromine, trazodone, trifluoperazine, trimipramine, tryptophan, valerian, valproic acid in 2.3:1 ratio, varenicline, venlafaxine, vilazodone, vortioxetine, zaleplon, ziprasidone, zolpidem, zopiclone, zotepine, zuclopenthixol and pharmaceutically-acceptable salts of any of these.

Opioid analgesics that may be employed in formulations of the invention include buprenorphine, butorphanol, codeine, fentanyl, hydrocodone, hydromorphone, meperidine, methadone, morphine, nomethadone, opium, oxycodone, oxymorphone, pentazocine, tapentadol, tramadol and pharmaceutically-acceptable salts of any of these.

Opioid antagonists that may be employed in formulations of the invention include naloxone, nalorphine, niconalorphine, diprenorphine, levallorphan, samidorphan, nalodeine, alvimopan, methylnaltrexone, naloxegol, 60-naltrexol, axelopran, bevenopran, methylsamidorphan, naidemedine, preferably nalmefene and, especially, naltrexone, as well as pharmaceutically-acceptable salts of any of these.

Anticancer agents that may be included in formulations of the invention include actinomycin, afatinib, all-trans retinoic acid, amsakrin, anagrelid, arseniktrioxid, axitinib, azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, dasatinib, daunorubicin, decitabine, docetaxel, doxifluridine, doxorubicin, epirubicin, epothilone, erlotinib, estramustin, etoposide, everolimus, fludarabine, fluorouracil, gefitinib, guadecitabine, gemcitabine, hydroxycarbamide, hydroxyurea, idarubicin, idelalisib, ifosfamide, imatinib, irinotecan, ixazomib, kabozantinib, karfilzomib, krizotinib, lapatinib, lomustin, mechlorethamine, melphalan, mercaptopurine, mesna, methotrexate, mitotan, mitoxantrone, nelarabin, nilotinib, niraparib, olaparib, oxaliplatin, paclitaxel, panobinostat, pazopanib, pemetrexed, pixantron, ponatinib, procarbazine, regorafenib, ruxolitinib, sonidegib, sorafenib, sunitinib, tegafur, temozolomid, teniposide, tioguanine, tiotepa, topotecan, trabektedin, valrubicin, vandetanib, vemurafenib, venetoklax, vinblastine, vincristine, vindesine, vinflunin, vinorelbine, vismodegib, as well as pharmaceutically-acceptable salts of any of these.

Such compounds may be used in any one of the following cancers: adenoid cystic carcinoma, adrenal gland cancer, amyloidosis, anal cancer, ataxia-telangiectasia, atypical mole syndrome, basal cell carcinoma, bile duct cancer, Birt-Hogg Dub4, tube syndrome, bladder cancer, bone cancer, brain tumor, breast cancer (including breast cancer in men), carcinoid tumor, cervical cancer, colorectal cancer, ductal carcinoma, endometrial cancer, esophageal cancer, gastric cancer, gastrointestinal stromal tumor, HER2-positive, breast cancer, islet cell tumor, juvenile polyposis syndrome, kidney cancer, laryngeal cancer, acute lymphoblastic leukemia, all types of acute lymphocytic leukemia, acute myeloid leukemia, adult leukemia, childhood leukemia, chronic lymphocytic leukemia, chronic myeloid leukemia, liver cancer, lobular carcinoma, lung cancer, small cell lung cancer, Hodgkin's lymphoma, non-Hodgkin's lymphoma, malignant glioma, melanoma, meningioma, multiple myeloma, myelodysplastic syndrome, nasopharyngeal cancer, neuroendocrine tumor, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, polycythemia vera, prostate cancer, renal cell carcinoma, retinoblastoma, salivary gland cancer, sarcoma, Kaposi sarcoma, skin cancer, small intestine cancer, stomach cancer, testicular cancer, thymoma, thyroid cancer, uterine (endometrial) cancer, vaginal cancer, Wilms' tumor.

Cancers that may be mentioned include myelodysplastic syndrome and sub-types, such as acute myeloid leukemia, refractory anemia or refractory anemia with ringed sideroblasts (if accompanied by neutropenia or thrombocytopenia or requiring transfusions), refractory anemia with excess blasts, refractory anemia with excess blasts in transformation, and chronic myeloid (myelomonocytic) leukemia leukemia.

Other drugs that may be mentioned for use in formulations of the invention include immunomodulatory imide drugs, such as thalidomide and analogues thereof, such as pomalidomide, lenalidomide and apremilast, and pharmaceutically-acceptable salts of any of these. Other drugs that many be mentioned include angiotensin II receptor type 2 agonists, such as Compound 21 (C21; 3-[4-(1H-imidazol-1-ylmethyl)phenyl]-5-(2-methylpropyl)thiophene-2-[(N-butyloxylcarbamate)-sulphonamide] and pharmaceutically-acceptable (e.g. sodium) salts thereof.

Preferred anticancer agents include lenalidomide, which is useful in the treatment of multiple myeloma and anaemia in low to intermediate risk myelodysplastic syndrome and, especially, azacitidine, which is useful in the treatment of certain subtypes of myelodysplastic syndrome.

Other preferred biologically-active agents that may be mentioned include liraglutide, which is useful in the treatment of type 2 diabetes mellitus and prevention of cardiovascular complications associated with diabetes.

Alternatively, formulations as described herein may also comprise, instead of (or in addition to) biologically-active agents, diagnostic agents (i.e. agents with no direct therapeutic activity per se, but which may be used in the diagnosis of a condition, such as a contrast agents or contrast media for bioimaging).

Formulations of the invention may include one or more of any of the aforementioned biologically active agents, particularly in view of the fact that any component, or combination of components, of a formulation of the invention (including the coatings or carrier system) may cause an inflammatory response after injection, e.g. subcutaneously.

However, biologically active agents that may in particular be mentioned include those in which the biologically active agent may, on its own or in the form of a formulation of the invention, produce an inflammatory response when administered to a patient, or may be expected to produce such a response.

In this respect, biologically active agents that may in particular be mentioned for use in formulations of the invention include, for example, antineoplastic agents, topoisomerase inhibitors, immunomodulators (such as thalidomide, pomalidomide, lenalidomide and apremilast), immunostimulants, immunosuppressants, chemotherapeutics, growth factors, vasodilators and radiopharmaceuticals.

Particular biologically active agents that may be mentioned in this regard include any one or more of the specific anticancer agents listed above and, in particular, actinomycin, azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, daunorubicin, decitabine, docetaxel, doxifluridine, doxorubicin, epirubicin, epothilone, estramustin, etoposide, everolimus, fludarabine, fluorouracil, guadecitabine, gemcitabine, hydroxyurea, idarubicin, ifosfamide, irinotecan, Ixazomib, karfilzomib, lomustin, mechlorethamine, melphalan, mercaptopurine, mesna, methotrexate, mitotan, mitoxantrone, nelarabin, oxaliplatin, paclitaxel, panobinostat, pemetrexed, pixantron, procarbazine, tegafur, temozolomide, teniposide, tioguanine, tiotepa, topotecan, trabektedin, valrubicin, venetoclax, vinblastine, vincristine, vindesine, vinflunine and vinorelbine, as well as pharmaceutically acceptable salts of any of these.

Further biologically active agents that may be mentioned in this respect include certain cytokines, proteins, and vaccines, as well as therapeutic peptides/proteins such as daratumumab and isatuximab.

Other drugs that may be mentioned in this regard include bendamustine, bleomycin, carboplatin, chlorambucil, cisplatin, cyclophosphamide, cyclosporin, cytarabine, dacarbazine, dactinomycin, daunorubicin, docetaxel, doxorubicin, epirubicin, etoposide, everolimus, fluorouracil, gemcitabine, ifosfamide, irinotecan, mercaptopurine, mesna, methotrexate, midazolam, mitomycin, oxaliplatin, paclitaxel, procarbazine, temsirolimus, thioguanine, vinblastine, vincristine, vinorelbine or pharmaceutically acceptable salts of any of these. A specific drug that may be mentioned is cisplatin.

Non-biologically active adjuvants, diluents and carriers that may be employed in cores to be coated in accordance with the relevant aspects of the invention may include pharmaceutically-acceptable substances that are soluble in water, such as carbohydrates, e.g. sugars, such as lactose and/or trehalose, and sugar alcohols, such as mannitol, sorbitol and xylitol; or pharmaceutically-acceptable inorganic salts, such as sodium chloride. Preferred carrier/excipient materials include sugars and sugar alcohols. Such carrier/excipient materials are particularly useful when the biologically-active agent is a complex macromolecule, such as a peptide, a protein or portions of genetic material or the like, for example as described generally and/or the specific peptides/proteins described hereinbefore including vaccines. Embedding complex macromolecules in excipients in this way will often result in larger cores for coating, and therefore larger coated particles.

In a preferred embodiment of this aspect of the invention, the cores as described hereinbefore are provided in the form of nanoparticles or, more preferably, microparticles. Preferred weight-, number-, or volume-based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. The person skilled in the art will also understand there are other suitable ways of expressing mean diameters, such as area based mean diameters, and that these other expressions of mean diameter are interchangeable with those used herein. Other instruments that are well known in the field may be employed to measure particle size, such as equipment sold by e.g. Malvern Instruments, Ltd (Worcestershire, UK) and Shimadzu (Kyoto, Japan).

Particles may be spherical, that is they possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, the coating of particles on any shape is also possible in accordance with this aspect of the invention. For example, irregular shaped (e.g. ‘raisin’-shaped), needle-shaped, flake-shaped or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the invention.

Particles may be obtained in a form in which they are suitable to be coated or be obtained in that form, for example by particle size reduction processes (e.g. crushing, cutting, milling or grinding) to a specified weight based mean diameter (as hereinbefore defined), for example by wet grinding, dry grinding, air jet-milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, freeze-drying, spray-freeze-drying, vacuum-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, crystallization, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for e.g. ALD precursors.

Preferred methods of applying the coating(s) to the cores comprising biologically-active agents in accordance with the aforementioned preferred aspect of the invention include gas phase techniques, such as ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

Coating materials that may be applied to said cores may be pharmaceutically-acceptable, in that they should be essentially non-toxic.

Coating materials may comprise organic or polymeric materials, such as a polyamide, a polyimide, a polyurea, a polyurethane, a polythiourea, a polyester or a polyimine.

Coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

Inorganic coating materials may comprise one or more metals or metalloids, or may comprise one or more metal-containing, or metalloid-containing, compounds, such as metal, or metalloid, oxides, nitrides, sulphides, selenides, carbonates, and/or other ternary compounds, etc. Metal, and metalloid, hydroxides and, especially, oxides are preferred, especially metal oxides.

Metals that may be mentioned include alkali metals, alkaline earth metals, noble metals, transition metals, post-transition metals, lanthanides, etc. Metal and metalloids that may be mentioned include aluminium, titanium, magnesium, iron, gallium, zinc, zirconium, niobium, hafnium, tantalum, lanthanum, and/or silicon; more preferably aluminium, titanium, magnesium, iron, gallium, zinc, zirconium, and/or silicon; especially aluminium, silicon, titanium and/or zinc.

As mentioned above, the formulations of the invention may comprise two or more discrete layers of (e.g. Inorganic) coating materials, the nature and chemical composition(s) of those layers may differ from layer to layer.

Individual layers may also comprise a mixture of two or more inorganic materials, such as metal oxides or metalloid oxides, and/or may comprise multiple layers or composites of different inorganic or organic materials, to modify the properties of the layer.

Coating materials that may be mentioned include those comprising aluminium oxide (Al₂O₃), titanium dioxide (TiO₂), iron oxides (FeₓO_{y}, e.g. FeO and/or Fe₂O₃ and/or Fe₃O₄), gallium oxide (Ga₂O₃), magnesium oxide (MgO), zinc oxide (ZnO), niobium oxide (Nb₂O₅), hafnium oxide (HfO₂), tantalum oxide (Ta₂O₅), lanthanum oxide (La₂O₃), zirconium dioxide (ZrO₂) and/or silicon dioxide (SiO₂). Preferred coating materials include aluminium oxide, titanium dioxide, iron oxides, gallium oxide, magnesium oxide, zinc oxide, zirconium dioxide and silicon dioxide. More preferred coating materials include iron oxide, titanium dioxide, zinc sulphide, more preferably zinc oxide, silicon dioxide and/or aluminium oxide.

Layers of coating materials (on an individual or a collective basis) In coated cores of said relevant formulations of the invention may consist essentially (e.g. may be greater than about 80%, such as greater than about, 90%, e.g. about 95%, such as about 98%) of iron oxides, titanium dioxide, or more preferably zinc oxide, silicon oxide and/or aluminium oxide.

The processes described herein are particularly useful when the coating material(s) that is/are applied to the cores comprise zinc oxide, silicon dioxide and/or aluminium oxide.

It is further preferred that the inorganic coating material comprises zinc oxide, and more particularly a mixture of:
(i) zinc oxide (ZnO); and
(ii) one or more other metal and/or metalloid oxides,
   wherein the atomic ratio ((i):(ii)) is between at least about 1:10 (e.g. about 1:6) and up to and including about 10:1 (e.g. about 6:1).

It Is preferred that the atomic ratio ((i):(ii)) is between at least about 1:1 and up to and including about 6:1 The coating of comprising a mixture of zinc oxide and one or more other metal and/or metalloid oxides is referred to hereinafter as a ‘mixed oxide’ coating or coating material(s).

The biologically active agent-containing cores may thus be coated with a coating material that comprises a mixture of zinc oxide, and one or more other metal and/or metalloid oxides, at a atomic ratio of zinc oxide to the other oxide(s) that is at least about 1:10 (e.g. at least about 1:6, including at least about 1:4, such as at least about 1:2), preferably at least about 1:1 (e.g. at least about 1.5:1, such as at least about 2:1), including at least about 2.25:1, such as at least about 2.5:1 (e.g. at least about 3.25:1 or least about 2.75:1 (Including 3:1)), and is up to (i.e. no more than) and including about 10:1, such as about 6:1, including up to about 5.5:1, or up to about 5:1, such as up to about 4.5:1, including up to about 4:1 (e.g. up to about 3.75:1).

In ALD, in most instances, the first of the consecutive reactions will involve some functional group or free electron pairs or radicals at the surface to be coated, such as a hydroxy group (—OH) or a primary or secondary amino group (—NH₂ or —NHR where R e.g. Is an aliphatic group, such as an alkyl group). The individual reactions are advantageously carried out separately and under conditions such that all excess reagents and reaction products are essentially removed before conducting the subsequent reaction.

Thus, when ALD is employed, the above-described mixed oxide coating may be prepared by feeding a first, zinc-, other metal- or metalloid-containing precursor into an ALD reactor chamber (in a so called ‘precursor pulse’) to form the adsorbed atomic or molecular zinc-, other metal- or metalloid-containing monolayer at the surface of the particle. A second precursor (e.g. water) Is then pulsed into the reactor and reacts with the first precursor, resulting in the formation of a monolayer of zinc, metal or metalloid oxide, respectively, on the substrate surface. A subsequent purging pulse is followed by a further pulse of the first precursor, and thus the start of a new cycle of the same events, which is an ALD cycle.

In order to make a mixed oxide coating with an atomic ratio of (for example) between about 1:1 and up to and including about 6:1 of zinc oxide relative to the one or more other metal and/or metalloid oxides, the skilled person will appreciate that for every one ALD cycle (i.e. monolayer) of the other oxide(s), between about 1 and about 6 ALD cycles of zinc oxide must also be deposited. For example, for a 3:1 atomic (zinc:other oxide) mixed oxide coating to be formed, 3 zinc-containing precursor pulses may each be followed by second precursor pulses, forming 3 monolayers of zinc oxide, which will then be followed by 1 pulse of the other metal and/or metalloid-containing precursor followed by second precursor pulse, forming 1 monolayer of oxide of the other metal and/or metalloid. Alternatively, 6 monolayers of zinc oxide may be followed by 2 monolayers of the other oxide, or any other combination so as to provide an overall atomic ratio of about 3:1. In this respect, the order of pulses to produce the relevant oxides is not critical, provided that the resultant atomic ratio is in the relevant range in the end.

When such mixed oxide coatings are employed, the other metal or metalloid oxide material preferably comprises one or other or both of aluminium oxide (Al₂O₃) and/or silicon dioxide (SiO₂).

There is provided a method of preparing of plurality of coated particles in accordance with the invention, wherein the coated particles are made by applying precursors of at least two metal and/or metal oxides forming a mixed oxide on the solid cores, and/or previously-coated solid cores, by a gas phase deposition technique. Precursors for forming a metal oxide or a metalloid oxide often include an oxygen precursor, such as water, oxygen, ozone and/or hydrogen peroxide; and a metal and/or metalloid compound, typically an organometal compound or an organometalloid compound.

Non-limiting examples of precursors are as follows: Precursors for zinc oxide may be water and diC₁-C₅alkylzinc, such as diethylzinc. Precursors for aluminium oxide may be water and triC₁-C₅alkylaluminium, such as trimethylaluminium. Precursors for silicon oxide (silica) may be water as the oxygen precursor and silanes, alkylsilanes, aminosilanes, and orthosilicic acid tetraethyl ester. Precursors for iron oxide includes oxygen, ozone and water as the oxygen precursor; and di C₁-C₅alkyl-iron, dicyclopropyl-iron, and FeCl₃. It will be appreciated that the person skilled in the art is aware of what precursors are suitable for the purpose as disclosed herein.

In ALD, layers of coating materials may be applied at process temperatures from about 20° C. to about 800° C., or from about 40° C. to about 200° C., e.g. from about 40° C. to about 150° C., such as from about 50° C. to about 100° C. The optimal process temperature depends on the reactivity of the precursors and/or substances (including biologically-active agents) that are employed in the core and/or melting point of the core substance(s). It is preferred that a lower temperature, such as from about 30° C. to about 100° C. is employed. In particular, in one embodiment of the method a temperature from about 20° C. to about 80° C. is employed, such as from about 30° C. to about 70° C., such as from about 40° C. to about 60° C., such as about 50° C.

We have found that, when coatings comprising zinc oxide are applied using ALD at a lower temperature, such as from about 50° C. to about 100° C., unlike other coating materials, such as aluminium oxide and titanium oxide, that form amorphous layers, the coating materials are largely crystalline in their nature.

Without being limited by theory, because zinc oxide is crystalline, if only zinc oxide is employed as coating material, we are of the understanding that interfaces may be formed between adjacent crystals of zinc oxide that are deposited by ALD, through which a carrier system, medium or solvent in which zinc oxide is partially soluble (e.g. an aqueous solvent system) can ingress following suspension therein. It is believed that this may give rise to dissolution that is too fast for the depot-forming composition that it is intended to make.

In addition, previous studies have shown that, when suspended in aqueous media, the relative bioavailability for formulations comprising an active ingredient that has been coated with zinc oxide is lower than uncoated active ingredient. We believe that this lower relative bioavailability is due to degradation of the active ingredient before it can be released into systemic circulation. Penetration of water through crystalline interfaces within a zinc oxide coating as described above is thought to lead to hydrolysis of the active ingredient within the interior of the coated particle.

These problems may be alleviated by making a mixed oxide coating as described herein. In particular, by forming a mixed oxide coating as described herein, that is predominantly, but not entirely, comprised of zinc oxide, we have been able to coat active ingredients with coatings that appear to be essentially amorphous, or a composite between crystalline and amorphous material and/or in which ingress of injection vehicles such as water may be reduced. In this respect, it appears to us that the presence of the aforementioned perceived interfaces may be reduced, or avoided altogether, by employing the mixed oxide aspect of the invention, in either a heterogeneous manner (in which the other oxide is ‘filling in’ gaps formed by the interfaces), or in a homogeneous manner (In which a true composite of mixed oxide materials is formed during deposition, in a manner where the interfaces are potentially avoided in the first place).

The gas phase deposition reactor chamber used may optionally, and/or preferably, be a stationary gas phase deposition reactor chamber. The term ‘stationary’, in the context of gas phase deposition reactor chambers, will be understood to mean that the reactor chamber remains stationary while in use to perform a gas phase deposition technique, excluding negligible movements and/or vibrations such as those caused by associated machinery for example.

Additionally, a so-called ‘stop-flow’ process may be employed. Using a stop-flow process, once the first precursor has been fed into the reactor chamber and prior to the first precursor being purged from the reactor chamber, the first precursor may be allowed to contact the cores in the reactor chamber for a pre-determined period of time (which may considered as a soaking time). During the pre-determined period of time there is preferably a substantial absence of pumping that may result in flow of gases and/or a substantial absence of mechanical agitation of the cores.

The employment of the stop-flow process may increase coating uniformity by allowing each gas to diffuse conformally in high aspect-ratio substrates, such as powders. The benefits may be even more pronounced when using precursors with slow reactivity as more time is given for the precursor to react on the surface. This may be evident especially when depositing mixed oxide coatings according to the invention. For example, when depositing a mixed zinc oxide/aluminium oxide coating as described herein, we have found that a zinc-containing precursor, such as diethylzinc (DEZ), which has a lower reaction probability towards the surface of a substrate than, for example, aluminium containing precursors, such as trimethylaluminum (TMA).

In addition to generating coatings with good shell integrity and more controlled release profiles, the employment of such a stop-flow process may improve the ability to achieve a particular coating composition.

For example, when attempting to employ a gas phase technique to produce a coating comprising an atomic ratio of 3:1 between zinc and aluminium in the resulting shell as described above, we have found that a ratio that is much closed to 3:1 may be achieved using a stop-flow process than when depositing material using a continuous flow of precursors.

Preferably, and/or optionally, a ‘multi-pulse’ technique may also be employed to feed the first precursor, the second precursor or both precursors to the reactor chamber.

Using such a multi-pulse technique, the respective precursor may be fed into the reactor chamber as a plurality of ‘sub-pulses’, each lasting a short period of time such as 1 second up to about a minute (depending on the size and the nature of the gas phase deposition reactor), rather than as one continuous pulse. The precursor may be allowed to contact the cores in the reactor chamber for the pre-determined period of time, for example from about 1 to 500 seconds, about 2 to 250 seconds, about 3 to 100 seconds, about 4 to 50 seconds, or about 5 to 10 seconds, for example 9 seconds, after each sub-pulse. Again, depending on the size and the nature of the gas phase deposition reactor, this time could be extended up to several minutes (e.g. up to about 30 minutes). The introduction of a sub-pulse followed by a period of soaking time may be repeated a pre-determined number of times, such as between about 5 to 1000 times, about 10 to 250 times, or about 20 to 50 times in a single step.

Preferably, more than one separate layers or coating material (also referred to herein as ‘coatings’ or ‘shells’, all of which terms are used herein interchangeably) are applied (that is ‘separately applied’) to the solid cores comprising the biologically active agent sequentially.

By ‘separate application’ of ‘separate layers, coatings or shells’, we mean that the solid cores are coated with a first layer of coating material, and then that resultant coated core is subjected to some form of deagglomeration process. In this respect, the number of discrete layers of coating material(s) as defined herein corresponds to the number of these intermittent deagglomeration steps with a final mechanical deagglomeration being conducted prior to the application of a final layer of coating material.

In other words, ‘gas-phase deposition (e.g. ALD) cycles’ can be repeated several times to provide a ‘gas-phase deposition (e.g. ALD) set’ of cycles, which may consist of e.g. 10, 25 or 100 cycles. However, after this set of cycles, the coated core is subjected to some form of deagglomeration step, which is then followed by a further set of cycles.

This process may be repeated as many times as is desired and, in this respect, the number of discrete layers of coating material(s) as defined herein corresponds to the number of these intermittent deagglomeration steps with a final mechanical deagglomeration being conducted prior to the application of a final layer (set of cycles) of coating material.

The terms ‘disaggregation’ and ‘deagglomeration’ are used interchangeably when referring to the coated particles, and disaggregating coated particles aggregates is preferably done by way of a mechanical sieving technique.

Coated cores may be removed from the coating apparatus, such as the ALD reactor, and thereafter subjected to an external deagglomeration step, for example as described in International patent application WO 2014/187995. Such an external deagglomeration step may comprise agitation, such as sonication in the wet or dry state, or preferably may comprise subjecting the resultant solid product mass that has been discharged from the reactor to sieving, e.g. by forcing it through a sieve or mesh in order to deagglomerate the particles, for example as described hereinafter, prior to placing the particles back into the coating apparatus for the next coating step. Again, this process may be continued for as many times as is required and/or appropriate prior to the application of the final coating.

In an external deagglomeration process, deagglomeration may alternatively be effected (additionally and/or instead of the abovementioned processes) by way of subjecting the coated particles in the wet or dry state to one or more of nozzle aerosol generation, milling, grinding, stirring, high sheer mixing and/or homogenization. If the step(s) of deagglomeration are carried out on particles in the wet state, the deagglomerated particles should be dried (as hereinbefore described in relation to cores) prior to the next coating step.

However, we prefer that, in such an external process, the deagglomeration step(s) comprise one or more sieving step(s), which may comprise jet sieving, manual sieving, vibratory sieve shaking, horizontal sieve shaking, tap sieving, or (preferably) sonic sifting as described hereinafter, or a like process, including any combination of these sieving steps. Manufacturers of suitable sonic sifters include Advantech Manufacturing, Endecott and Tsutsui.

It is further preferred that at least one of the mechanical sieving steps comprises a vibrational sieving technique.

Such a vibrational sieving technique comprises a vibration motor coupled to a sieve, and may provide a means of vibrationally forcing the solid product mass formed by coating said cores through a sieve that may be located internally or (preferably) externally to (i.e. outside of) the reactor, and is configured to deagglomerate any particle aggregates upon said vibrational forcing of the coated cores, prior to being subjected to a second and/or a further layer of coating material. This process is repeated as many times as is required and/or appropriate prior to the application of a final layer of coating material.

Vibrational forcing means comprises a vibration motor which is coupled to a sieve. The vibration motor is configured to vibrate and/or gyrate when an electrical power is supplied to it. For example, the vibration motor may be a piezoelectric vibration motor comprising a piezoelectric material which changes shape when an electric field is applied, as a consequence of the converse piezoelectric effect. The changes in shape of the piezoelectric material cause acoustic or ultrasonic vibrations of the piezoelectric vibration motor.

The vibration motor may alternatively be an eccentric rotating mass (ERM) vibration motor comprising a mass which is rotated when electrical power is supplied to the motor. The mass is eccentric from the axis of rotation, causing the motor to be unbalanced and vibrate and/or gyrate due to the rotation of the mass. Further, the ERM vibration motor may comprise a plurality of masses positioned at different locations relative to the motor. For example, the ERM vibration motor may comprise a top mass and a bottom mass each positioned at opposite ends of the motor. By varying each mass and its angle relative to the other mass, the vibrations and/or gyrations of the ERM vibration motor can be varied.

The vibration motor is coupled to the sieve in a manner in which vibrations and/or gyrations of the motor when electrical power is supplied to it are transferred to the sieve.

The sieve and the vibration motor may be suspended from a mount (such as a frame positionable on a floor, for example) via a suspension means such that the sieve and motor are free to vibrate relative to the mount without the vibrations being substantially transferred to or dampened by the mount. This allows the vibration motor and sieve to vibrate and/or gyrate without impediment and also reduces noise generated during the vibrational sieving process. The suspension means may comprise one or more springs or bellows (i.e. air cushion or equivalent cushioning means) that couple the sieve and/or motor to the mount. Manufacturers of vibratory sieves or sifters suitable for carrying out such a process include for instance Russell Finex, SWECO, filtra Vibracion, VibraScreener, Gough Engineering and Farley Greene.

Preferably, the vibrational sieving technique further comprises controlling a vibration probe coupled to the sieve. The vibration probe may be controlled to cause the sieve to vibrate at a separate frequency to the frequency of vibrations caused by the vibration motor. Preferably the vibration probe causes the sieve to vibrate at a higher frequency than the vibrations caused by the vibration motor and, more preferably, the frequency is within the ultrasonic range.

Providing additional vibrations to the sieve by means of the vibration probe reduces the occurrence of clogging in the sieve, reduces the likelihood of the sieve being overloaded and decreases the amount of time needed to clean the mesh of the sieve.

Preferably, the aforesaid vibrational sieving technique comprises sieving coated particles with a throughput of at least 1 g/minute. More preferably, the vibrational sieving technique comprises sieving coated particles with a throughput of 4 g/minute or more.

The throughput depends on the area of the sieve mesh, mesh-size of the sieve, the particle size, the stickiness of the particles, static nature of the particle. By combining some of these features a much higher throughput is possible. Accordingly, the vibrational sieving technique may more preferably comprise sieving coated particles with a throughput of up to 1 kg/minute or even higher.

Any one of the above-stated throughputs represents a significant improvement over the use of known mechanical sieving, or sifting, techniques. For example, we found that sonic sifting involved sifting in periods of 15 minutes with a 15-minute cooling time in-between, which is necessary for preserving the apparatus. To sift 20 g of coated particles required 9 sets of 15 minutes of active sifting time, i.e. a total time (including the cooling) of 255 minutes. By comparison, by using the aforementioned vibrational sieving technique, 20 g of coated particles may be sieved continuously in, at most, 20 minutes, or more preferably in just 5 minutes, or less.

The sieve mesh size may be determined so that the ratio of the size of the sieved or sonic sifted particles to the sieve mesh size is about 1:>1, preferably about 1:2, and optionally about 1:4. The size mesh size may range from about 20 μm to about 100 μm, preferably from about 20 μm to about 60 μm.

Appropriate sieve meshes may include perforated plates, microplates, grid, diamond, threads, polymers or wires (woven wire sieves) but are preferably formed from metals, such as stainless steel.

Surprisingly, using a stainless steel mesh within the vibrational sieving technique is as gentle to the particle coatings as using a softer polymer sieve as part of a mechanical sieving technique such as sonic sifting.

Also, a known problem with sieving powders is the potentially dangerous generation of static electricity. A steel mesh has the advantage of removing static electricity from the powder while that is not the case with a polymeric mesh, which has to be used in a sonic sifter.

Further, the mesh size of known sonic sifters is limited to about 100 μm since the soundwaves travel through the mesh rather than vibrating it. That limitation does not exist using for vibrational sieving techniques as there is no reliance on soundwaves to generate vibrations in the sieve. Therefore, the vibrational sieving technique described herein allows larger particles to be sieved than if alternative mechanical sieving techniques were used.

If a (e.g. vibrational) sieve is located externally to (i.e. outside of) the reactor, the process for making coated cores of formulations of the invention comprises discharging the coated particles from the gas phase deposition reactor prior to subjecting the coated particles to agitation, followed by reintroducing the deagglomerated, coated particles into the gas phase deposition reactor prior to applying a further layer of at least one coating material to the reintroduced particles.

We have found that applying separate layers of coating materials following external deagglomeration gives rise to visible and discernible interfaces that may be observed by analysing coated particles according to the invention, and are observed by e.g. TEM as regions of higher electron permeability. In this respect, the thickness of the layers between interfaces correspond directly to the number of cycles in each series that are carried out within the ALD reactor, and between individual external agitation steps.

Because, in an ALD coating process, coating takes place at the atomic level, such clear, physical interfaces are typically more difficult to observe.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants or the core material, such as an API forming part of the core, that do not originate from the coating process, such as ALD.

In the alternative, coated cores may be subjected to the aforementioned deagglomeration process internally, without being removed from said apparatus by way of a continuous process. Such a process preferably involves forcing solid product mass formed by coating said cores through a sieve that is located within the reactor, and is configured to deagglomerate any particle aggregates upon forcing of the coated cores by means of a forcing means applied within said reactor, prior to being subjected to a second and/or a further coating. This process is continued for as many times as is required and/or appropriate prior to the application of the final coating as described herein.

Having a deagglomeration step (such as a sieve) located within the reactor vessel means that the coating can be applied by way of a continuous process which does not require the particles to be removed from the reactor. Thus, no manual handling of the particles is required, and no external machinery is required to deagglomerate the aggregated particles. This not only considerably reduces the time of the coating process being carried out, but is also more convenient and reduces the risk of harmful (e.g. poisonous) materials being handled by personnel. It also enhances the reproducibility of the process by limiting the manual labour and reduces the risk of contamination.

Whether carried out inside or outside of the reactor, particle aggregates are preferably broken up by a forcing means that forces them through a sieve, thus separating the aggregates into individual particles or aggregates of a desired and predetermined size (and thereby achieving deagglomeration). In the latter regard, in some cases the individual primary particle size is so small (i.e. <1 μm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ In the form of small aggregate particles.

In this way, the term ‘particles’, when referring to the particles that have been deagglomerated and coated in the context of the invention, refers to both individual (primary) particles and aggregate (secondary) particles of a desired size.

In any event, the desired particle size (whether that be of individual particles or aggregates of a desired size) Is maintained and, moreover, continued application of the gas phase coating mechanism to the particles after such deagglomeration via the sieving means that a complete coating is formed on the particle, thus forming fully coated particles (individual or aggregates of a desired size).

Whether carried out inside or outside of the reactor, the above-described repeated coating and deagglomeration process may be carried out at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and 20 times, e.g. between 3 and 15 times, such as 10 times, e.g. 9 or 8 times, more preferably 6 or 7 times, and particularly 4 or 5 times.

Whether carried out inside or outside of the reactor, it is preferred that at least one sieving step is carried out and further that that step preferably comprises a vibrational sieving step as described above. It is further preferred that at least the final sieving step comprises a vibrational sieving step being conducted prior to the application of a final layer (set of cycles) of coating material. However, it is further preferred that more than one (including each) of the sieving steps comprise vibrational sieving techniques, steps or processes as described herein.

The preferable repetition of these steps makes the improved throughput of any vibrational sieving technique all the more beneficial.

The total thickness of the coating (meaning all the separate layers/coatings/shells) will on average be in the region of between about 0.5 nm and about 2 μm.

The minimum thickness of each individual layer/coating/shell will on average be in the region of about 0.1 nm (including about 0.5 nm, for example about 0.75 nm, such as about 1 nm).

The maximum thickness of each individual layer/coating/shell will depend on the size of the core (to begin with), and thereafter the size of the core with the coatings that have previously been applied, and may be on average about 1 hundredth of the mean diameter (i.e. the weight-, number-, or volume-based mean diameter) of that core, or core with previously-applied coatings.

Preferably, for particles with a mean diameter that is between about 100 nm and about 1 μm, the total coating thickness should be on average between about 1 nm and about 5 nm; for particles with a mean diameter that is between about 1 μm and about 20 μm, the coating thickness should be on average between about 1 nm and about 10 nm; for particles with a mean diameter that is between about 20 μm and about 700 μm, the coating thickness should be on average between about 1 nm and about 100 nm.

We have found that applying coatings/shells followed by conducting one or more deagglomeration step such as sonication gives rise to abrasions, pinholes, breaks, gaps, cracks and/or voids (hereinafter ‘cracks’) in the layers/coatings, due to coated particles essentially being more tightly ‘bonded’ or ‘glued’ together directly after the application of a thicker coating. This may expose a core comprising biologically-active ingredient to the elements once deagglomeration takes place.

As it is intended to provide particles in a suspension prior to administration to a patient, it is necessary to provide deagglomerated primary particles without pinholes or cracks in the coatings. Such cracks will result in an undesirable initial peak (burst) in plasma concentration of active ingredient directly after administration.

We have found that, by conducting one or more of the deagglomeration steps described herein, this gives rise to significantly less pinholes, gaps or cracks in the final layer of coating material, giving rise to particles that are not only completely covered by that layer/coating, but are also covered in a manner that enables the particles to be deagglomerated readily (e.g. using a non-aggressive technique, such as vortexing) in a manner that does not destroy the layers of coating material that have been formed, prior to, and/or during, pharmaceutical formulation.

In this respect, the (e.g. inorganic, such as mixed oxide) coating typically completely surrounds, encloses and/or encapsulates said solid cores comprising active ingredient(s). In this way, the risk of an initial drug concentration burst due to the drug coming into direct contact with solvents in which the relevant active ingredient is soluble is minimized. This may include not only bodily fluids, but also any medium in which such coated particles may be suspended prior to injection.

Thus in a further embodiment of this aspect of the invention, there are provided particles as hereinbefore disclosed, wherein said coating surrounding, enclosing and/or encapsulating said core covers at least about 50%, such as at least about 65%, including at least about 75%, such as at least about 80%, more particularly at least about 90%, such as at least about 91%, such as at least about 92%, such as at least about 93%, such as at least about 94%, such as at least about 95%, such as at least about 96%, such as at least about 97%, such as at least about 98%, such as at least about 99%, such as approximately, or about, 100%, of the surface of the solid core, such that the coating essentially completely surrounds, encloses and/or encapsulates said core.

As used herein, the term ‘essentially completely coating completely surrounds, encloses and/or encapsulates said core’ means a covering of at least about 98%, or at least about 99%, of the surface of the solid core.

In the alternative, processes described herein may result in the deagglomerated coated particles with the essential absence of said cracks through which active ingredient can be released in an uncontrolled way

Although some minor cracks may appear in the said coating without effecting the essential function thereof in terms of controlling release, in a further embodiment, there are provided particles as hereinbefore disclosed, wherein at least about 90% of the particles do not exhibit cracks in the coating surrounding, enclosing and/or encapsulating said core. In one embodiment at least about 91%, such as at least about 92%, such as at least about 93%, such as at least about 94%, such as at least about 95%, such as at least about 96%, such as at least about 97%, such as at least about 98%, such as at least about 99%, such as approximately 100% of the particles do not exhibit said cracks.

Alternatively, by ‘essentially free of said cracks’ in the coating(s), we also mean that less than about 1% of the surfaces of the coated particles comprise abrasions, pinholes, breaks, gaps, cracks and/or voids through which active ingredient is potentially exposed (to, for example, the elements).

The layers of coating material may, taken together, be of an essentially uniform thickness over the surface area of the particles. By ‘essentially uniform’ thickness, we mean that the degree of variation in the thickness of the coating of at least about 10%, such as about 25%, e.g. about 50%, of the coated particles that are present in a formulation of the invention, as measured by TEM, is no more than about ±20%, including 50%, of the average thickness.

Different coating materials, such as pharmaceutically-acceptable and essentially non-toxic coating materials may also be applied in addition, either between separate coatings as described herein (e.g. In-between separate deagglomeration steps) and/or whilst a particular coating is being applied. Such materials may comprise multiple layers or composites of said mixed oxide and one or more different inorganic or organic materials, to modify the properties of the layer(s).

Although the plurality of coated particles according to the invention are essentially free of the aforementioned cracks in the applied coatings, through which active ingredient is potentially exposed (to, for example, the elements), two further, optional steps may be applied to the plurality of coated particles prior to subjecting it to further pharmaceutical formulation processing.

The first optional step may comprise, subsequent to the final deagglomeration step as hereinbefore described, application of a final overcoating layer, the thickness of which outer ‘overcoating’ layer/coating, or ‘sealing shell’ (which terms are used herein interchangeably), must be thinner than the previously-applied separate layers/coatings/shells (or ‘subshells’).

The thickness may therefore be on average no more than a factor of about 0.7 (e.g. about 0.6) of the thickness of the widest previously-applied subshell. Alternatively, the thickness may be on average no more than a factor of about 0.7 (e.g. about 0.6) of the thickness of the last subshell that is applied, and/or may be on average no more than a factor of about 0.7 (e.g. about 0.6) of the average thickness of all of the previously-applied subshells. The thickness may be on average in the region of about 0.3 nm to about 10 nm, for particles up to about 20 μm. For larger particles, the thickness may be on average no more than about 1/1000 of the coated particles' weight-, number-, or volume-based mean diameter.

The role of such as sealing shell is to provide a ‘sealing’ overcoating layer on the particles, covering over those cracks, so giving rise to particles that are not only completely covered by that sealing shell, but also covered in a manner that enables the particles to be deagglomerated readily (e.g. using a non-aggressive technique, such as vortexing) in a manner that does not destroy the subshells that have been formed underneath, prior to, and/or during, pharmaceutical formulation.

For the reasons described herein, it is preferred that the sealing shell does not comprise zinc oxide. The sealing shell may on the other hand comprise silicon dioxide or, more preferably, aluminium oxide.

The second optional step may comprise ensuring that the few remaining particles with broken and/or cracked shells/coatings are subjected to a treatment in which all particles are suspended in a solvent in which the active ingredient is soluble (e.g. with a solubility of at least about 0.1 mg/mL), but the least soluble material in the coating is insoluble (e.g. with a solubility of no more than about 0.1 μg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed hereinbefore.

At the end of the process, coated particles may be dried using one or more of the techniques that are described hereinbefore for drying cores. Drying may take place in the absence, or in the presence, of one or more pharmaceutically-acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘Intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of formulations of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al., J. Mater. Chem., 18, 3650 (2008) and U.S. Pat. No. 8,097,742). Another compound, e.g. a desired targeting compound may be attached to such an anchoring group by way of covalent bonding, or non-covalent bonding, including hydrogen bonding, or van der Waals bonding, or a combination thereof.

The presence of such anchoring groups may provide a versatile tool for targeted delivery to specific sites in the body. Alternatively, the use of compounds such as PEG may cause particles to circulate for a longer duration in the blood stream, ensuring that they do not become accumulated in the liver or the spleen (the natural mechanism by which the body eliminates particles, which may prevent delivery to diseased tissue).

Formulations of the invention can for example be used in medicine, diagnostics, and/or in veterinary practice.

Pharmaceutical (or veterinary) formulations of the invention may include particles of different types, for example particles comprising different active ingredients, comprising different functionalization (as described hereinbefore), particles of different sizes, and/or different thicknesses of the layers of coating materials, or a combination thereof. By combining, in a single pharmaceutical formulation, particles with different coating thicknesses and/or different core sizes, the drug release following administration to patient may be controlled (e.g. varied or extended) over a specific time period.

Formulations of the invention may be administered systemically, for example by Injection or Infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transdermally, or intraperitoneally, in the form of a pharmaceutically-(or veterinarily) acceptable dosage form. As stated above, preferred administration routes include intratumorally, more preferably subcutaneously and/or intramuscularly.

The preparation of formulation of the invention comprises incorporation of active ingredients (including coated particles) as described herein into an appropriate pharmaceutically- or veterinarily-acceptable carrier system, and may be achieved with due regard to the intended route of administration and standard pharmaceutical practice. Thus, appropriate carrier systems should be chemically inert to the biologically-active agent(s) that is/are employed, and have no detrimental side effects or toxicity under the conditions of use. Such pharmaceutically-acceptable carriers may also impart an immediate, or a modified, release of biologically active agent from the particles of the formulations of the invention.

In order to form depot compositions following subcutaneous, intratumoral and/or intramuscular injection, more preferably subcutaneous and/or intramuscular injection, formulations of the invention may be in the form of sterile injectable and/or infusible dosage forms, for example, sterile aqueous or oleaginous suspensions of formulations of the invention.

Formulations of the invention that comprise an aqueous carrier system may be formulated as sterile aqueous suspensions of particles (coated or otherwise), in accordance with techniques known in the art. The aqueous media should contain at least about 50% water, but may also comprise other aqueous excipients, such as Ringer's solution, and may also include polar co-solvents (e.g. ethanol, glycerol, propylene glycol, 1,3-butanediol, polyethylene glycols of various molecular weights and tetraglycol); viscosity-Increasing, or thickening, agents (e.g. carboxymethylcellulose, microcrystalline cellulose, hydroxypropylmethyl cellulose, hydroxyethyl cellulose, ethyl hydroxyethyl cellulose, sodium starch glycolate, Poloxamers, such as Poloxamer 407, polyvinylpyrrolidone, cyclodextrins, such as hydroxypropyl-β-cyclodextrin, polyvinylpyrrolidone and polyethylene glycols of various molecular weights); surfactant/wetting agents to achieve a homogenous suspension (e.g. sorbitan esters, sodium lauryl sulfate; monoglycerides, polyoxyethylene esters, polyoxyethylene alkyl ethers, polyoxylglycerides and, preferably, Tweens (Polysorbates), such as Tween 80 and Tween 20). Preferred ingredients include isotonicity-modifying agents (e.g. sodium lactate, dextrose and, especially, sodium chloride); pH adjusting and/or buffering agents (e.g. citric acid, sodium citrate, and especially phosphate buffers, such as disodium hydrogen phosphate dihydrate, sodium acid phosphate, sodium dihydrogen phosphate monohydrate and combinations thereof, which may be employed in combination with standard inorganic acids and bases, such as hydrochloric acid and sodium hydroxide); as well as other ingredients, such as mannitol, croscarmellose sodium and hyaluronic acid.

In the alternative, oleaginous, or oil-based carrier systems may comprise one or more pharmaceutically- or veterinarily-acceptable liquid lipid, which may include fixed oils, such as mono-, di- or triglycerides, including miglyol (e.g. 812N), propylene glycol dicaprylocaprate (Miglyol 840, C8/C10 esters), tricaprylin (Miglyol oil), gelucire 43/01, kollisolv GTA, labrafil. The carrier systems may also comprise polysorbates, such as polysorbate 20, polysorbate 60, polysorbate 80, glycols, such as propylene glycol, polyethylene glycol, polyethylene glycol 300, polyethylene glycol 400, polyethylene glycol 600, and/or natural and/or refined pharmaceutically-acceptable oils, such as olive oil, peanut oil, soybean oil, corn oil, cottonseed oil, sesame oil, castor oil, oleic acid, and their polyoxyethylated versions (e.g. sorbitan trioleate, lauroglycol 90, capryol PGMC, PEG-60 hydrogenated castor oil, polyoxyl 35 castor oil). More preferred carrier systems include mono-, di- and/or triglycerides, wherein most preferred is medium chain triglycerides, such as alkyl chain triglycerides (e.g. C₆-C₁₂ alkyl chain triglycerides).

Such injectable suspensions may be formulated in accordance with techniques that are well known to those skilled in the art, by employing suitable dispersing or wetting agents (e.g. Tweens, such as Tween 80), and suspending agents.

Formulations of the invention may further be formulated in the form of injectable suspension of (e.g. coated) particles with a size distribution that is both even and capable of forming a stable suspension within the injection liquid (i.e. without settling) and may be injected through a needle.

In this respect, the formulations of the invention may comprise a medium that is viscous enough to prevent sedimentation, leading to suspensions that are not ‘homogeneous’ and thus the risk of under or overdosing of active ingredient. For any given plurality of coated particles, this can be achieved via the addition of known viscosity modifying agents (as hereinbefore described) or, more preferably, by providing a more viscous carrier system per se.

Formulations of the invention may be stored under normal storage conditions, and maintain their physical and/or chemical integrity.

The phrase ‘maintaining physical and chemical integrity’ essentially means chemical stability and physical stability.

By ‘chemical stability’, we include that any formulation of the invention may be stored (with or without appropriate pharmaceutical packaging), under normal storage conditions, with an insignificant degree of chemical degradation or decomposition. The term ‘chemical stability’ also includes ‘stereochemical’ and/or ‘configurational’ stability, by which we mean resistance to stereochemical conversion, such as racemisation, at one or more chiral centres within a molecule of an active ingredient.

By ‘physical stability’, we include that the any formulation of the invention may be stored (with or without appropriate pharmaceutical packaging), under normal storage conditions, with an insignificant degree of physical transformation, such as sedimentation as described above, or changes in the nature and/or integrity of the coated particles, for example in the coating itself or the active ingredient (including dissolution, solvatisation, solid state phase transition, etc.).

Examples of ‘normal storage conditions’ for formulations of the invention include temperatures of between about −50° C. and about +80° C. (preferably between about −25° C. and about +75° C., such as about 50° C.), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, formulations of the invention may be found to be less than about 15%, more preferably less than about 10%, and especially less than about 5%, chemically and/or physically degraded/decomposed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Formulations of the invention may be in the form of a liquid, a sol or a gel, which is administrable via a surgical administration apparatus, e.g. a needle, a catheter or the like, to form a depot formulation.

In any event, the preparation of suitable formulations may be achieved non-inventively by the skilled person using routine techniques. Formulations of the invention and dosage forms comprising them, may thus be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al., ‘The Theory and practice of Industrial Pharmacy’, Lea & Febiger, 3^{rd} edition (1986); ‘Remington: The Science and Practice of Pharmacy’, Troy (ed.), University of the Sciences in Philadelphia, 21^{st} edition (2006); and/or ‘Aulton's Pharmaceutics: The Design and Manufacture of Medicines’, Aulton and Taylor (eds.), Elsevier, 4^{th} edition, 2013), and the documents referred to therein, the relevant disclosures in all of which documents are hereby incorporated by reference.

Antiinflammatory agents that may be employed in formulations of the invention include butylpyrazolidines (such as phenylbutazone, mofebutazone, oxyphenbutazone, clofezone, kebuzone and suxibuzone); acetic acid derivatives and related substances (indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac and bufexamac); oxicams (such as piroxicam, tenoxicam, droxicam, lornoxicam and meloxicam); propionic acid derivatives (such as ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, vedaprofen, carprofen and tepoxalin); fenamates (such as mefenamic acid, tolfenamic acid, flufenamic acid, meclofenamic acid and flunixin), coxibs (such as celecoxib, rofecoxib, valdecoxib, parecoxib, etoricoxib, lumiracoxib, firocoxib, robenacoxib, mavacoxib and cimicoxib); other non-steroidal antiinflammatory agents (such as nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, pentosan polysulfate and aminopropionitrile); corticosteroids (such as 11-dehydrocorticosterone, 11-deoxycorticosterone, 11-deoxycortisol, 11-ketoprogesterone, 11β-hydroxypregnenolone, 11β-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone (prebediolone), aldosterone, corticosterone (17-deoxycortisol), cortisol (hydrocortisone), cortisone, pregnenolone, progesterone, flugestone (flurogestone), fluorometholone, medrysone (hydroxymethylprogesterone), prebediolone acetate (21-acetoxypregnenolone), chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol (halobetasol), amcinonide, budesonide, ciclesonide, deflazacort, desonide, formocortal fluclorolone acetonide (flucloronide), fludroxycortide (flurandrenolone, flurandrenolide), flunisolide, fluocinolone acetonide, fluocinonide, halcinonide and triamcinolone acetonide); quinolines (such as oxycinchophen); gold preparations (such as sodium aurothiomalate, sodium aurothiosulfate, auranofin, aurothioglucose and aurotioprol); penicillamine and similar agents (such as bucillamine); and antihistamines (such as akrivastin, alimemazine, antazolin, astemizol, azatadin, azelastin, bamipin, bilastin, bromdifenhydramin, bromfeniramin, buklizin, cetirizin, cinnarizine, cyklizin, cyproheptadine, deptropine, desloratadin, dexbromfeniramin, dexklorfeniramin, difenylpyralin, dimenhydrinat, dimetinden, doxylamin, ebastin, epinastin, fenindamin, feniramin, fexofenadin, histapyrrodin, hydroxietylprometazin, isotipendyl, karbinoxamin, ketotifen, kifenadin, klemastin, klorcyklizin, klorfenamin, klorfenoxamin, kloropyramin, levocetirizin, loratadin, mebhydrolin, mekitazin, meklozin, mepyramin, metapyrilen, metdilazin, mizolastin, oxatomide, oxomemazine, pimetixen, prometazin, pyrrobutamin, rupatadin, sekifenadin, talastin, tenalidin, terfenadin, tiazinam, tietylperazin, tonzylamin, trimetobenzamid, tripelennamin, triprolidine and tritokvalin). Combinations of any one or more of the above mentioned antiinflammatory agents may be used.

Preferred antiinflammatory agents include corticosteroids and non-steroidal anti-Inflammatory drugs, such as diclofenac, ketoprofen, meloxicam, aceclofenac, flurbiprofen, parecoxib, ketoralac tromethamine, indomethacin, or pharmaceutically-acceptable salts of any of these compounds. Preferred corticosteroids that may be used in accordance with the invention include hydrocortisone, triamcinolone and more preferably, methylprednisolone, prednisolone, dexamethasone, bethametasone, or pharmaceutically-acceptable salts of any of these compounds. Combinations of one or more of the above-mentioned corticosteroids and non-steroidal anti-inflammatory drugs may be used.

However, in formulations of the invention, biologically active agents are ‘combined’ with antiinflammatory agents, which means that the respective active ingredients are presented (i.e. formulated) as a combined preparation including both active agents, which are then administered together.

In formulations of the invention, the antiinflammatory agent may be co-presented with other biologically-active agent at an appropriate dose by:
(1) formulating it along with the biologically-active agent and the extended-release component (e.g. in form of solid cores within coated particles), and then suspending the resultant composition in the carrier system of the formulation of the invention (which formulation is hereinafter referred to as a ‘combined preparation’); or
(2) dissolving it, and/or suspending it, within the carrier system of a formulation of the invention (which formulation is hereinafter referred to as a ‘combination preparation’).

In embodiment (2) above, the antiinflammatory agent may be presented in a formulation of the invention in any form in which it is separate to the other components (e.g. cores) that contain the (other) biologically-active agent as hereinbefore described. This may be achieved by, for example, dissolving or suspending the antiinflammatory agent directly in the carrier system/vehicle that also forms part of a formulation of the invention, or by presenting it in a form in which its release can, like the (other) biologically-active agent, also be controlled following injection.

The latter option may be achieved by, for example, providing the antiinflammatory agent in a form in which it is combined with one or more extended-release components as described hereinbefore, more preferably in the form of (e.g. additional) particles suspended in the carrier system of formulation of the invention, which additional particles have a weight-, number-, or volume-based mean diameter that is between about 10 nm and about 700 μm, and comprise cores comprising that antiinflammatory agent, which cores are coated, at least in part, by one or more coating materials as hereinbefore described, which may allow for the release of the antiinflammatory agent over the same, or over a different, timescale (such formulations are hereinafter referred to as ‘combination suspensions’).

Although, in such combination suspensions, the coated cores comprising the antiinflammatory agent may be different in terms of their chemical composition(s) and/or physical form(s), it is preferred that the coating that is employed is the same or similar to that employed in formulations of the invention, for the reasons hereinbefore described.

This may mean that the antiinflammatory agent is coated with one or more inorganic coatings as hereinbefore described, for example one or more inorganic coating materials comprising one or more metal-containing, or metalloid-containing, compounds, such as a metal, or metalloid, oxide, for example iron oxide, titanium dioxide, zinc sulphide, more preferably zinc oxide, silicon dioxide and/or aluminium oxide, which coating materials may (on an individual or a collective basis) consist essentially (e.g. are greater than about 80%, such as greater than about, 90%, e.g. about 95%, such as about 98%) of such oxides, and more particularly inorganic coatings comprising a mixture of:
(i) zinc oxide; and
(ii) one or more other metal and/or metalloid oxides,
   wherein the atomic ratio ((i):(ii)) is at least about 1:10 (such as at least about) 1:6 and up to and including about 10:1 (such as about 6:1).

Preferably, the atomic ratio ((i):(ii)) is at least about 1:1 and up to and including about 6:1.

In accordance with this aspect of the invention, formulations may comprise between about 1% to about 99%, such as between about 10% (such as about 20%, e.g. about 50%) to about 90% by weight of the coated particles with the remainder made up by carrier system and/or other excipients.

According to a further aspect of the invention there is provided a process for the preparation of a formulation of the invention, which process comprises mixing the biologically-active ingredient together with said one or more extended-release component (e.g. to make coated particles containing biologically-active ingredient as described herein) with the antiinflammatory agent and the carrier system as described herein.

The above process may further comprise:
(A) making a combined preparation as hereinbefore defined, comprising the biologically-active ingredient, the antiinflammatory agent and the one or more extended-release component (e.g. In the form of coated cores as hereinbefore described containing both active ingredients); and/or
(B) separately providing a component comprising said biologically-active ingredient together with the one or more extended-release component (e.g. In the form of coated cores as hereinbefore described) and mixing this with said antiinflammatory agent (optionally in association with one or more (same of different) extended-release components, which may thus be in the form of particles that are uncoated or coated as hereinbefore described); and then
   mixing the above components (A) and/or (B) together with the carrier system as described herein, if necessary and/or appropriate, along with (e.g. further) antiinflammatory agent.

In a further embodiment of the invention there is provided a method of treatment of a medical condition in a patient, which method comprises:
(1) making a first injectable pharmaceutical or veterinary formulation comprising:
   (a) a biologically active agent in which medical condition said agent is useful;
   (b) an extended-release component that, following intratumoral or, more preferably, subcutaneous and/or intramuscular injection of that first formulation to a subject, forms a depot composition that provides for an extended release of said biologically active agent within a subject (for example a plurality of coated cores as defined herein); and
   (c) a carrier system as defined herein,
(2) injecting that first formulation intratumorally or, more preferably subcutaneously and/or intramuscularly (as appropriate to a subject; and
(3) if a localised inflammatory response is observed, administering an antiinflammatory agent to said patient in accordance with one or more of the principles described hereinbefore or hereinafter.

In two further aspects of the invention, there are provided:
a biologically active agent for use in a method of treating a medical condition in which said agent is useful, and
the use of a biologically active agent for the manufacture of a medicament for use in a method of treating a medical condition in which said agent is useful,
   which uses/methods comprise:
(1) making a first injectable pharmaceutical or veterinary formulation comprising:
   (a) a biologically active agent in which medical condition said agent is useful;
   (b) an extended-release component that, following intratumoral or, more preferably, subcutaneous and/or intramuscular injection of that first formulation to a subject, forms a depot composition that provides for an extended release of said biologically active agent within a subject (for example a plurality of coated cores as defined herein); and
   (c) a carrier system as defined herein,
(2) Injecting that first formulation intratumorally or, more preferably subcutaneously and/or intramuscularly (as appropriate) to a subject; and
(3) if a localised inflammatory response is observed, administering an antiinflammatory agent to said patient in accordance with one or more of the principles described hereinbefore or hereinafter.

In a preferred aspect of this embodiment of the invention, the administering of the antiinflammatory agent may further comprise the making of a second injectable pharmaceutical or veterinary formulation that comprises said antiinflammatory agent and an extended-release component (e.g. a plurality of coated cores comprising said antiinflammatory agent as defined herein) and a carrier system that, following, intratumoral or, preferably, subcutaneous or intramuscular injection of the formulation to a subject, forms a depot composition that provides for an extended release of said antiinflammatory agent within a subject to provide an antiinflammatory effect.

In this respect, said administering of the antiinflammatory agent may comprise injecting that second formulation intratumorally or, more preferably, subcutaneously and/or intramuscularly to a subject as part of the treatment of a condition that may be treated by said biologically active agent in the absence of said antiinflammatory agent.

Such administration of the antiinflammatory agent may take place at essentially the same time as (e.g. within about a minute of, or concomitantly with) administration of the biologically active agent, or may take place before (e.g. up to about 10 minutes prior to), or at any time after, said injection, on multiple occasions, in accordance with standard safety criteria.

In the alternative, said antiinflammatory agent may be applied in the form of a topical formulation to the surface of the skin around the point of injection. Such topical formulations comprising said antiinflammatory agents are commercially available, and/or may be made using routine techniques, in the form of e.g. creams, lotions, gels, mousses, ointments, tapes and bandages, solutions and the like) and may be applied before (e.g. up to about 10 minutes prior to) said injection, during (i.e. at essentially the same time as, e.g. within about a minute of, or concomitantly with) said injection, or at any time after said injection, on multiple occasions, in accordance with standard safety criteria. Preferred topical non-steroidal antiinflammatory compositions may include diclofenac, ibuprofen, diclofenac, eltenac, etoricoxib, felbinac, flufenamate, flurbiprofen, indomethacin, ibuprofen, ketoprofen, nimesulide, piketoprofen, and piroxicam. Preferred corticosteroid-based topical compositions may include clobetasone, hydrocortisone, beclomethasone, clobetasol, fluticasone and mometasone.

In the case where biologically active agent and antiinflammatory agent are administered concomitantly, a further embodiment there is provided a method of treatment of a medical condition in a patient, which method comprises:
(1) making an injectable pharmaceutical or veterinary formulation comprising:
   (a) a biologically active agent that gives, may give, or is expected to give, rise to localized inflammation when injected into, and exposed to tumoral or, more preferably muscular and/or subcutaneous tissue;
   (b) an antiinflammatory agent;
   (c) one or more extended-release component that, following intratumoral or, more preferably, subcutaneous or intramuscular injection (as appropriate) of the formulation to a subject, forms a depot composition that provides for an extended release of said biologically active agent and (optionally) said antiinflammatory agent within a subject; and
   (d) a carrier system as defined herein; and
(2) injecting that formulation intratumorally or, more preferably, subcutaneously or intramuscularly to a subject to treat a condition that may be treated by said biologically active agent in the absence of said antiinflammatory agent.

In two further aspects of the invention, there are provided:
a biologically active agent for use in a method of treating a medical condition in which said agent is useful, and
the use of a biologically active agent for the manufacture of a medicament for use in a method of treating a medical condition in which said agent is useful,
   which uses/methods comprise:
(1) making an injectable pharmaceutical or veterinary formulation comprising:
   (a) a biologically active agent that gives, may give, or is expected to give, rise to localized inflammation when injected into, and exposed to tumoral or, more preferably muscular and/or subcutaneous tissue;
   (b) an antiinflammatory agent;
   (c) one or more extended-release component that, following intratumoral or, more preferably, subcutaneous or intramuscular Injection (as appropriate) of the formulation to a subject, forms a depot composition that provides for an extended release of said biologically active agent and (optionally) said antiinflammatory agent within a subject; and
   (d) a carrier system as defined herein; and
(2) injecting that formulation intratumorally or, more preferably, subcutaneously or intramuscularly to a subject to treat a condition that may be treated by said biologically active agent in the absence of said antiinflammatory agent.

The term ‘agent that gives, may give, or is expected to give, rise to a localized inflammation’ is as defined hereinbefore.

For the avoidance of doubt in relation to this latter aspect of the invention said antiinflammatory agent:
may be combined along with said inflammatory active agent with said one or more extended-release component;
may be combined with one or more extended-release component separately to the inflammatory active agent; and/or
may not be combined with one or more extended-release component, prior to admixing with an injectable carrier.

Formulations of the invention may be presented in the form of sterile injectable and/or infusible dosage forms administrable via a surgical administration apparatus (e.g. a syringe with a needle for injection, a catheter or the like), to form a depot formulation.

There is further provided an injectable and/or infusible dosage form comprising a formulation of the invention, wherein said formulation is contained within a reservoir that is connected to, and/or is associated with, an injection or infusion means (e.g. a syringe with a needle for injection, a catheter or the like).

Alternatively, formulations of the invention may be stored prior to being loaded into a suitable injectable and/or infusible dosing means (e.g. a syringe with a needle for injection) or may even be prepared immediately prior to loading into such a dosing means.

Sterile injectable and/or infusible dosage forms may thus comprise a receptacle or a reservoir in communication with an injection or infusion means into which a formulation of the invention may be pre-loaded, or may be loaded at a point prior to use, or may comprise one or more reservoirs, within which coated particles of the formulation of the invention and the carrier system are housed separately, and in which admixing occurs prior to and/or during injection or infusion.

There is thus further provided a kit of parts comprising:
(1) the biologically-active ingredient in combination with the one or more extended-release components as hereinbefore described, and the antiinflammatory agent, optionally in combination with one or more extended-release components as hereinbefore described; and
(2) a carrier system of the formulation of the invention;
   along with instructions to the end user to admix component (1) with (2) (and optionally further antiinflammatory agent, formulated as described herein in respect of any aspect of the invention) prior to injection.

There is in addition provided a kit of parts comprising:
(1) the biologically-active ingredient in combination with the one or more extended-release components as hereinbefore described; and
(2) a carrier system of the formulation of the invention;
   along with instructions to the end user to:
(i) admix component (1) with (2) and prior to injection; and
(ii) If, following injection, a localised inflammatory response is observed, administering an antiinflammatory agent to said patient in accordance with one or more of the principles described hereinbefore or hereinafter.

There is further provided a kit of parts comprising:
(a) coated particles of a formulation of the invention (whether those particles contain the biologically-active ingredient, contain the antiinflammatory agent, or contain both such active ingredients, e.g. In a combined preparation as hereinbefore described); and
(b) a carrier system of the formulation of the invention; and
(c) If necessary and/or appropriate, antiinflammatory agent, which may be in the form of particles that are uncoated or coated as hereinbefore described,
   as well as a kit of parts comprising a coated particles of the formulation of the invention (as described under (a) above), along with instructions to the end user to admix those particles with a carrier system (b) (and optionally said antiinflammatory agent (c)) according to the invention.

There is further provided a pre-loaded injectable and/or infusible dosage form as described in above, but modified by comprising at least two chambers, within one of which chamber is located the coated particles of the formulation of the invention (as described under (1) or (a) in the paragraphs immediately above), and within the other of which is located the carrier system of the formulation of the invention (2) or (b) in the paragraphs immediately above, wherein admixing occurs prior to and/or during injection or infusion. In such a dosage form, antiinflammatory agent may optionally be included in either or both of those chambers.

In addition to the above, subjects may receive (or may already be receiving) one or more of the aforementioned antiinflammatory agents separate to a formulation of the invention, by which we mean receiving a prescribed dose of one or more of those antiinflammatory agents, prior to, in addition to, and/or following, treatment with either:
a formulation of the invention, or
a formulation that is, in all other respects, a formulation of the invention, provided that it does not comprise an antiinflammatory agent.

In the latter case, formulations comprising a biologically-active agent and an antiinflammatory agent are administered separately (simultaneously or sequentially) in different formulations.

There is thus further provided a method of treatment of a patient, which comprises administration of:
(A) a pharmaceutical formulation of the invention, or a formulation that is, in all other respects, a formulation of the invention, provided that it does not comprise an antiinflammatory agent; and
(B) a pharmaceutical formulation comprising an antiinflammatory agent in admixture with one or more a pharmaceutically- or veterinarily-acceptable excipients, such as a carrier system as hereinbefore defined or in accordance with the invention, in which method, Components (A) and (B) are each provided in a form that is suitable for administration in conjunction with each other (hereinafter referred to as ‘combination methods’).

In two further aspects of the invention, there are provided:
a biologically active agent for use in a method of treating a medical condition in which said agent is useful, and
the use of a biologically active agent for the manufacture of a medicament for use in a method of treating a medical condition in which said agent is useful,
   which uses/methods comprise a combination method as defined above.

Component (B) of the combination method described above may be different in terms its chemical composition and/or physical form from Component (A), for example it may be made by dissolving or suspending that antiinflammatory agent directly in a carrier system/vehicle that may be the same or different to that employed in a formulation of the invention, or by presenting it in a form in which its release can, like the (other) biologically-active agent, also be controlled following injection.

In this respect, Component (B) above may also be in a form that is essentially the same or at least similar to a formulation of the invention, or a formulation that is in all other respects is a formulation of the invention provided that it does not comprise an antiinflammatory agent, but is instead for example in the form of a plurality of particles suspended in a carrier system, which particles:
(a) have a weight-, number-, or volume-based mean diameter that is between about 10 nm and about 700 μm; and
(b) comprise solid cores comprising that antiinflammatory agent, which cores are coated, at least in part, by one or more coatings (e.g. Inorganic coatings) as hereinbefore described, for example one or more inorganic coating materials comprising one or more metal-containing, or metalloid-containing, compounds, such as a metal, or metalloid, oxide, for example iron oxide, titanium dioxide, zinc sulphide, more preferably zinc oxide, silicon dioxide and/or aluminium oxide, which coating materials may (on an individual or a collective basis) consist essentially (e.g. are greater than about 80%, such as greater than about, 90%, e.g. about 95%, such as about 98%) of such oxides, and more particularly inorganic coatings comprising a mixture of:
   (i) zinc oxide; and
   (ii) one or more other metal and/or metalloid oxides,
   wherein the atomic ratio ((i):(ii)) is at least about 1:10 (such as about 1:6) and up to and including about 10:1 (such as about 6:1).

Preferably, the atomic ratio ((i):(ii)) is at least about 1:1 and up to and including about 6:1.

In any event, and for the avoidance of doubt, all aspects, including preferred aspects, disclosed and/or claimed herein for formulations of the invention generally are equally applicable as aspects, and/or preferences for coated cores comprising one or more antiinflammatory agents described above, whether presented as a combined preparation, a combination preparation or a combination suspension, or as part of a combination method or combination pack. For the avoidance of doubt, such aspects, preferences and features, alone or in combination, are hereby incorporated by reference to these aspects of the invention.

According to a further aspect of the invention, there is provided a combination method as defined above, which method comprises bringing Component (A), as defined above, into association with a Component (B), as defined above, thus rendering the two components suitable for administration in conjunction with each other.

By bringing the two components ‘into association with’ each other, we include that Components (A) and (B) of the combination method may be:
(i) provided as separate formulations (i.e. Independently of one another), which are subsequently brought together for use in conjunction with each other in combination treatment; or
(ii) packaged and presented together as separate components of a ‘combination pack’ for use in conjunction with each other in combination treatment.

Thus, there is further provided a kit of parts comprising Components (A) and (B) of the combination method as hereinbefore defined packaged and presented together as separate components of a combination pack, for use in conjunction with each other in combination treatment, as well as a kit of parts comprising:
(I) one of Components (A) and (B) as defined herein; together with
(II) instructions to use that component in conjunction with the other of the two components

As alluded to above, the combination method described herein may comprise more than one formulation including an appropriate quantity/dose of biologically active agent, and/or more than one formulation including an appropriate quantity/dose of antiinflammatory agent, in order to provide for repeat dosing as hereinbefore described.

In this respect, with respect to the combination method as described herein, by ‘administration in conjunction with’, we include that Components (A) and (B) of the combination method are administered, sequentially, separately and/or simultaneously, over the course of treatment of a relevant condition.

Thus, the term ‘in conjunction with’ Includes that one or other of the two formulations may be administered (optionally repeatedly) prior to, after, and/or at the same time as, administration of the other component. When used in this context, the terms ‘administered simultaneously’ and ‘administered at the same time as’ Include that individual doses of the biologically active agent and antiinflammatory agent are administered within 48 hours (e.g. 24 hours) of each other.

In respect of any of the combination methods or products according to the invention, the respective formulations are administered optionally repeatedly, in conjunction with each other, in a manner that may enable a beneficial effect for the subject, that is greater, over the course of the treatment of a relevant condition, than if a formulation comprising only the relevant biologically active agent is administered (e.g. repeatedly, as described herein) in the absence of the antiinflammatory agent, over the same course of treatment.

Determination of whether a combination method or product provides a greater beneficial effect in respect of, and over the course of treatment will depend upon the condition to be treated and/or its severity, but may be achieved routinely by the skilled person.

For example, a physician may initially administer a formulation of the invention, or a formulation that is, in all other respects, a formulation of the invention, provided that it does not comprise an antiinflammatory agent, to treat a patient with a relevant condition, and then find that that person exhibits an inflammatory response (which may be caused by the active ingredient per se and/or by any other component of the formulation).

The physician may then administer one or more of:
Component (B) of a combination method or combination pack as described above,
a combined preparation,
a combination preparation, and/or
a combination suspension
   as described above, any of which comprises an antiinflammatory agent as hereinbefore described.

All formulations of the invention, including combined preparations, combination preparations, combination suspensions, and/or combination methods and combination packs according to the invention, may be used in human medicine. In particular, they may be used in any indication in the relevant biologically active agent in question is either approved for use in, or otherwise known to be useful in.

The biologically active agents and antiinflammatory agents that may be employed in formulations of the invention, including combined preparations, combination preparations, combination suspensions, and/or combination methods or combination packs, according to the invention, may be provided in the form of a (e.g. pharmaceutically-acceptable) salt, including any such salts that are known in the art and described for the drugs in question to in the medical literature, such as Martindale—The Complete Drug Reference, 38^{th} Edition, Pharmaceutical Press, London (2014) and the documents referred to therein (the relevant disclosures in all of which documents are hereby incorporated by reference).

Otherwise, pharmaceutically acceptable salts of biologically active agents include acid addition salts and base addition salts. Such salts may be formed by conventional means, for example by reaction of a free acid or a free base form of a biologically active compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a biologically active compound in the form of a salt with another counter-ion, for example using a suitable ion exchange resin.

Formulations of the invention may comprise a pharmacologically-effective amount of relevant biologically-active agents. The term ‘pharmacologically-effective amount’ refers to an amount of such active ingredient, which is capable of conferring a desired physiological change (such as a therapeutic effect) on a treated patient, whether administered alone or in combination with another active ingredient. Such a biological or medicinal response, or such an effect, in a patient may be objective (i.e. measurable by some test or marker) or subjective (i.e. the subject gives an indication of, or feels, an effect), and includes at least partial alleviation of the symptoms of the disease or disorder being treated, or curing or preventing said disease or disorder.

The amount of any active agent that may be employed in formulations of the invention, including combined preparations, combination preparations, combination suspensions, and/or combination methods and combination packs according to the invention, must be sufficient so exert its pharmacological effect in a relevant condition.

Doses of active ingredients that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the active ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

As administration of formulations of the invention may be continuous or intermittent (e.g. by bolus injection), dosages of such other active ingredients may also be determined by the timing and frequency of administration.

In any event, the medical practitioner, or other skilled person, will be able to determine routinely the actual dosage of any particular active ingredient, which will be most suitable for an individual patient, and doses of the relevant active ingredients mentioned above include those that are known in the art and described for the drugs in question to in the medical literature, such as Martindale—The Complete Drug Reference, 38^{th} Edition, Pharmaceutical Press, London (2014) and the documents referred to therein, the relevant disclosures in all of which documents are hereby incorporated by reference.

In this respect, formulations of the invention and the uses and methods described herein allow for the formulation of a large diversity of pharmaceutically-active compounds. Formulations of the invention may be used to treat effectively a wide variety of disorders depending on the biologically-active agent that is included.

The use of formulations of the invention may, in respect of the biologically-active ingredient that is included in that formulation, control dissolution rate and/or the pharmacokinetic profile by reducing any burst effect (as characterised by a concentration maximum shortly after administration) and/or by reducing Cmax in a plasma concentration-time profile (thus, increasing the length of release of biologically-active ingredient from that formulation).

The formulations and processes described herein may have the advantage that, in the treatment of a relevant condition with a particular biologically-active agent, they may be more convenient for the physician and/or patient than, be more efficacious than, be less toxic than, have a broader range of activity than, be more potent than, produce fewer side effects than, or that it may have other useful pharmacological properties over, any similar treatments that may be described in the prior art for the same active ingredient.

Wherever the word ‘about’ is employed herein, for example in the context of amounts (e.g. numbers, concentrations, dimensions (sizes and/or weights), doses, time periods, pharmacokinetic parameters, etc.), relative amounts (percentages, weight ratios, size ratios, atomic ratios, aspect ratios, proportions, factors, fractions, etc.), relative humidities, lux, temperatures or pressures, it will be appreciated that such variables are approximate and as such may vary by ±15%, such as ±10%, for example ±5% and preferably ±2% (e.g. f1%) from the numbers specified herein. This is the case even if such numbers are presented as percentages in the first place (for example ‘about 15%’ may mean ±15% about the number 10, which is anything between 8.5% and 11.5%).

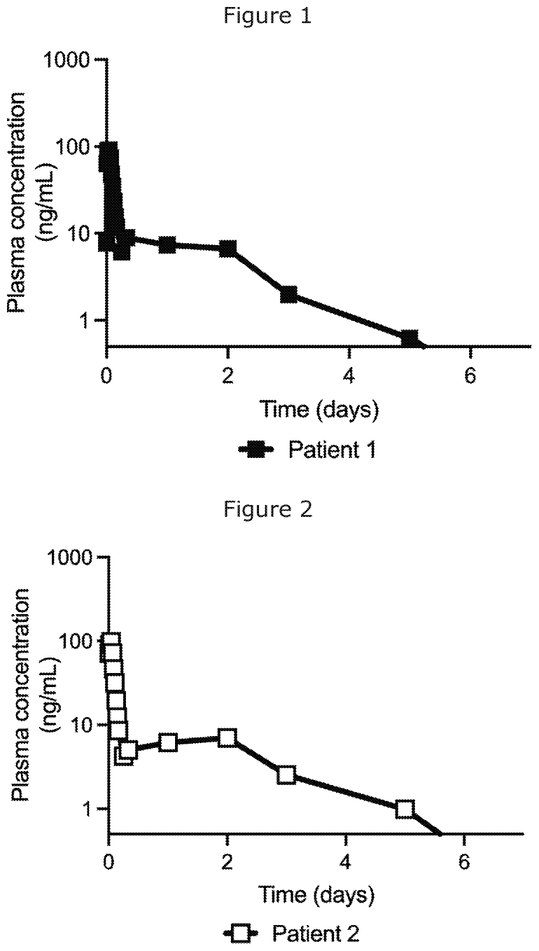

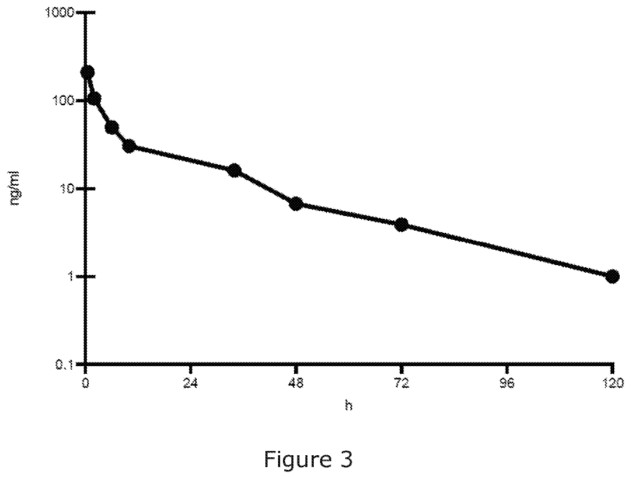

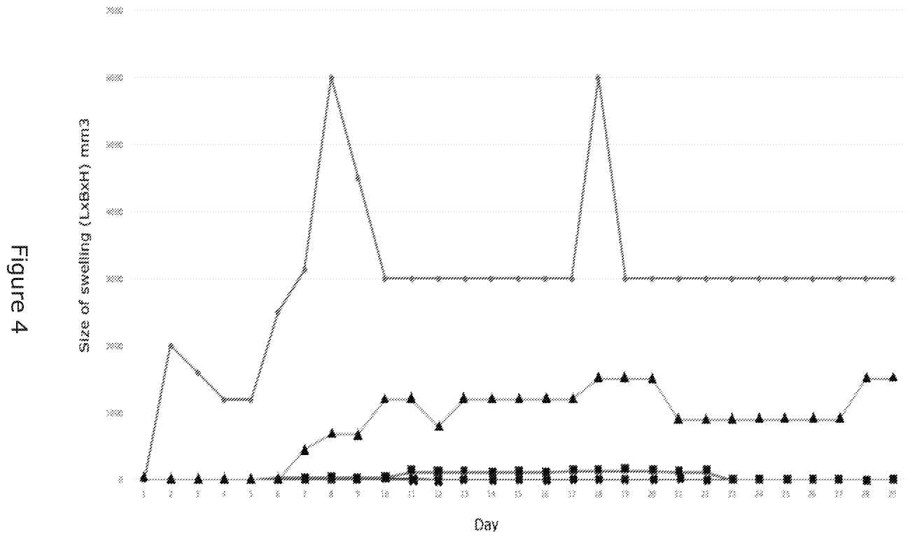

The invention is illustrated, but in no way limited, by the following examples, with reference to FIGS. 1 and 2, in which show plasma concentration-time profiles for two patients administered azacitidine according to a treatment protocol in a clinical trial as described in Comparative Example 2 below. FIG. 3 shows a plasma concentration-time profiles for a minipig following the subcutaneous administration of a formulation of the invention; and FIG. 4 shows the positive impact on local inflammatory response of subcutaneously co-administering formulations of the invention along with mixed oxide coated microparticles comprising the antiinflammatory agent, indomethacin.

### EXAMPLES

### Comparative Example 1

### Coated Azacitidine Microparticles

Samples of microparticles of azacitidine (MSN Labs, India) were prepared by jet-milling. The particle size distribution, as determined by laser diffraction, was as follows: D₁₀ 1.2 μm; D₅₀ 3.8 μm; D₅₀ 11.3 μm.

The powder was loaded to an ALD reactor (Picosun, SUNALE™ R-series, Espoo, Finland) where 24 ALD cycles were performed at a reactor temperature of 50° C. The coating sequence was three ALD cycles employing diethyl zinc and water as precursors for three ALD cycles, followed by one cycle of trimethylaluminium and water, repeated six times, to forming a mixed oxide layer of with an atomic ratio of zinc:aluminium of 3:1. The first layer was between about 4 and about 8 nm in thickness (as estimated from the number of ALD cycles).

The powder was removed from the reactor and deagglomerated by means of forcing the powder through a polymeric sieve with a 20 μm mesh size using a sonic sifter.

The resultant deagglomerated powder was re-loaded into the ALD reactor and further 24 ALD cycles were performed as before, forming a second layer of mixed oxide at the aforementioned ratio, followed by extraction from the reactor and deagglomeration by means of sonic sifting as above, followed by reloading to form a third layer, deagglomeration and then reloading to form a final, fourth layer.

To determine the drug load (i.e. w/w % of azacitidine in the powder), HPLC (Prominence-i (Shimadzu, Japan) equipped with a diode array detector (Shimadzu, Japan) set at 223 nm was employed using a 4.6×250 mm, 3 μm particles, C18 column (Luna, Phenomenex, USA)). The nanoshell coatings were dissolved in 5 M phosphoric acid in DMSO and the slurry was diluted with DMSO, before filtration (0.2 μm RC, Lab Logistics Group, Germany) and further analyzed with HPLC (n=2). The drug load was determined as 81.3%.

### Comparative Example 2

### Phase Ia Clinical Trial

An open pilot Phase Ia clinical study to assess the pharmacokinetics, tolerability, and safety of the coated azacitidine microparticles from Comparative Example 1 above suspended in Hyonate vet (Boehringer Ingelheim Animal Health; an aqueous solution comprising sodium hyaluronate (10 mg/mL), sodium chloride (8.5 mg/mL), disodium phosphate (0.223 mg/mL), sodium dihydrogen monohydrate phospate (40 μg/mL), HCl and NaOH for pH adjustment) and administered as a subcutaneous injection for the treatment of intermediate 2 or higher-risk MDS, CMML, or AML, in patients already on treatment with azacitidine, was carried out.

Pharmacokinetic parameters including AUC₀₋₂₄ₕ, AUC₀₋ₗₐₛₜ, AUC_{0-∞}), Cₘₐₓ, Cₗₐₛₜ, terminal t_{1/2}, volume of distribution V_{d} and clearance were measured.

Local tolerance were measured by inspection of injection sites. Pain, tenderness erythema/redness, and induration/swelling were assessed by a four-grade scale, in which 1 is considered mild and 4 is considered potentially life threatening.

It was intended to include 6 patients in the study, which would consist of a screening phase, a treatment phase, interim analysis, and a follow-up phase.

Inclusion criteria included:
Written Informed consent prior to any study specific procedures.
patients ≥18 years of age
Body Mass Index (BMI) ≥19 and ≤32 kg/m² BSA at screening
current treatment with azacitidine corresponding to 100 mg/m² BSA×5 or ×4 per treatment cycle for at least six cycles for diagnosed:
   a. Intermediate-2 and high-risk myelodysplastic syndromes (MDS) according to the International Prognostic Scoring System (IPSS)
   b. chronic myelomonocytic leukemia (CMML) with 10-29% marrow blasts
   c. acute myeloid leukemia (AML) according to World Health Organization (WHO) classification
Eastern Cooperative Oncology Group (ECOG) performance status of 0, 1, or 2
Recovery of Hematology and Clin. Chemistry assessment according to clinical practices at the start of the last azacitidine treatment cycle before the screening visit
Female subjects of non-childbearing potential (defined as pre-menopausal females with a documented tubal ligation or hysterectomy or bilateral oophorectomy; or as post-menopausal females defined as 12 months' amenorrhea)
Male patients agreed to use an adequate method of contraception
Willingness and ability to comply with study procedures, visit schedules, study restrictions, and requirements

### Exclusion Criteria Included:

That the patient has participated in any other investigational/interventional trial including an investigational drug within 30 days (or five half-lives of the study drug prior to screening, whichever is longer) prior to screening
Diagnosis of malignant disease within the previous 5 years (excluding basal cell carcinoma of the skin without complications, in-situ carcinoma of the cervix or breast, or other local malignancy excised or irradiated with a high probability of cure)
Any significant medical condition, laboratory abnormality, or psychiatric illness that would prevent the patient from participating in the study
History of alcohol abuse or drug abuse within the past 12 months
Any condition including the presence of laboratory abnormalities, which places the patient at unacceptable risk if he/she were to participate in the study
Other reasons for non-suitability for participation, as judged by the Investigator.

The duration for a patient in the study was intended to be approximately 2-3 months. This timeframe consisted of a 3-4 week screening period, followed by approximately four weeks in the treatment phase, which comprised, from Day 1 to Day 4, of daily injections of uncoated azacitidine (Vidaza® or generic azacitidine (Mylan), freeze dried powder for injection suspended in water for injection), both 100 mg/m² BSA, 25 mg/mL).

Samples were taken for pharmacokinetic analysis on Day 4 (before commencement of study drug). The mean maximum plasma concentration (Cₘₐₓ) was 562 ng/mL and occurred after a tₘₐₓ of 0.433 hours. The mean half-life was 6.82 hours. The mean AUC_{inf} was 1120 ng h/mL.

On Day 5, a single administration of the study drug suspension as described above was given (100 mg/m² BSA, 100 mg/mL). Samples were to be taken for pharmacokinetic analysis on each of Days 5 to 8, and then Days 10, 12, 15, 17 and 19.

It was also intended that, after the treatment phase, the last azacitidine dose would be replaced by a single dose of an azacitidine comparator (as above), and a follow-up visit scheduled to take place on the same day.

However, after two enrolled patients were subjected to the treatment phase, the study was put on hold after a meeting of an internal safety committee, which was charged with the review of safety data.

It was noted that induration and inflammation (redness and light pain), classified as moderate at the injection site was exhibited in both patients. It was decided not to enroll any more patients in the study and to request additional expert review/analysis(es) of biopsy results from the two patients.

The plasma concentration time curves for the two patients after administration of study drug are nevertheless presented in FIGS. 1 and 2, respectively, showing a clear steady-state sustained release of azacitidine from the injected study drug formulations. The plasma concentration time curve is presented in the graphs on a semi-logarithmic scale (squares). The mean maximum plasma concentration (Cₘₐₓ) was 94.8 ng/mL and occurred after (Tₘₐₓ) 1.02 hours. The mean half-life was 15.2 hours. The mean AUC_{inf} was 495 ng h/mL.

### Example 1

### Combination Formulations of the Invention

Various formulations of the invention comprising combinations of azacitidine and indomethacin are prepared as follows:

(A) A blended mixture of microparticles of azacitidine and indomethacin in a weight-ratio between 100:1 to 1:10, are prepared by jet-milling. The particle size distribution, as determined by laser diffraction, is an average particle size of between 0.1 and 100 μm.

The resultant powder is coated by ALD as described in Comparative Example 1 above and formulated in a vehicle and used for treatment of patients suffering from MDS as described in Comparative Example 2 above.

(B) Microparticles comprising an e.g. co-precipitated mixture of azacitidine and indomethacin in a weight-ratio between 100:1 to 1:10, are prepared. The particle size distribution, as determined by laser diffraction, is an average particle size of between 0.1 and 100 μm.

The microparticles are coated by ALD as described in Comparative Example 1 above and formulated in a vehicle and used for treatment of patients suffering from MDS as described in Comparative Example 2 above.

(C) Two sets of microparticle samples are separately prepared by jet-milling. A first set comprises azacitidine and a second set comprises indomethacin. The particle size distribution in both sets of samples, as determined by laser diffraction, is between 0.1 and 100 μm.

Both sets of samples are coated separately by ALD as described in Comparative Example 1 above and are mixed in a formulation wherein the weight-ratio between powders of the first and the second set is between 100:1 to 1:10.

The mixed powder is formulated in a vehicle and used for treatment of patients suffering from MDS as described in Comparative Example 2 above.

(D) Samples of azacitidine is prepared as described in Comparative Example 1 above are formulated in a vehicle as described in Comparative Example 2 above. An additional formulation comprising indomethacin particles is prepared in the same way.

Both formulations are used for injection at essentially the same time on different sites as described in Comparative Example 2 above for treatment of patients suffering from MDS. The dose ratio with respect to weight of azacitidine and indomethacin in the two different injections is between 100:1 and 1:10.

(E) Coated particles of azacitidine are prepared essentially as described in Comparative Example 1 above and are formulated in a vehicle as described in Comparative Example 2 above, further comprising dissolved and/or suspended indomethacin.

The formulation is used for treatment of patients suffering from MDS as described in Comparative Example 2 above.

In all of cases (A) to (E) above, any inflammatory reactions at the site of the subcutaneous administration (and formed depot) are suppressed by the antiinflammatory properties of indomethacin.

### Comparative Example 3

### Mixed Oxide Coated Azacitidine Microparticles II

The same procedure as described in Example 1 was conducted to produce coated azacitidine microparticles with a drug load that was determined as 80.1%.

### Comparative Example 4

### Mixed Oxide Coated Azacitidine Microparticles III

Essentially the same procedure as described in Example 1 was conducted, except that 30 ALD cycles were performed at a reactor temperature of 50° C., with a coating sequence of two ALD cycles employing diethyl zinc and water as precursors followed by one cycle of trimethylaluminium and water, repeated ten times, to forming a mixed oxide layer of with a atomic ratio of zinc:aluminium of 2:1. The first layer was estimated as between about 5 and about 10 nm in thickness.

The powder was removed from the reactor and deagglomerated by means of forcing the powder through a polymeric sieve with a 20 μm mesh size using a sonic sifter and then the deagglomerated powder re-loaded into the ALD reactor and a further 30 ALD cycles performed as above, forming a second layer of mixed oxide at the same ratio, extraction from the reactor and deagglomeration by using sonic sifting as above, with the process being repeated to form a total of eight layers.

The drug load was determined as 69.1%.

### Comparative Example 5

### Mixed Oxide Coated Indomethacin Microparticles

Samples of microparticles of indomethacin (Recce Pharmaceuticals, Australia) were prepared by jet-milling. The particle size distribution, as determined by laser diffraction, was as follows: D₁₀ 1.2 μm; D₅₀ 3.8 μm; D₉₀ 11.3 μm.

The same ALD coating and intermittent deagglomeration process as described in Example 1 was conducted to form coated indomethacin microparticles with four separate mixed oxide layers with a atomic ratio of zinc:aluminium of 3:1.

The drug load was determined as 80.1%.

### Comparative Example 6

### Mixed Oxide Coated Lactose Microparticles

Samples of microparticles of lactose (InhaLac® 400, Meggle, Germany) was used. The nominal particle size distribution was as follows: D₁₀ 0.8-1.6 μm; D₅₀ 4.0-11.0 μm; D₉₀ 15-35.0 μm.

The powder was loaded to an ALD reactor (Picosun, SUNALE™ R-series, Espoo, Finland) where 48 ALD cycles were performed at a reactor temperature of 50° C. The coating sequence was three ALD cycles employing diethyl zinc and water as precursors for three ALD cycles, followed by one cycle of trimethylaluminium and water, repeated twelve times, to forming a mixed oxide layer of with a atomic ratio of zinc:aluminium of 3:1. The first layer was between about 8 and about 16 nm in thickness (as estimated from the number of ALD cycles).

The powder was removed from the reactor and deagglomerated by means of forcing the powder through a polymeric sieve with a 20 μm mesh size using a sonic sifter.

The resultant deagglomerated powder was re-loaded into the ALD reactor and further ALD cycles were performed as before, forming a second layer of mixed oxide at the aforementioned ratio, followed by extraction from the reactor.

The particle size distribution of the coated lactose microparticles, as determined by laser diffraction, was as follows: D₁₀ 2.1 μm; D₅₀ 7.6 μm; D₉₀ 23.4 μm.

### Example 2

### Combination Formulations of the Invention II

Coated microparticles from Comparative Example 3 above, along with coated indomethacin microparticles from Comparative Example 5 above, were suspended together in Hyonate vet in a glass vial to give a final concentration of each coated active ingredient as set out in Table 1 below, in which the formulations are also identified in the manner they are referred to hereinafter.

TABLE 1

Coated Microparticles Concentration (mg/mL)

| Azacitidine | Indomethacin | Formulation |
|---|---|---|
| 100 | 25 | F |
| 100 | 5 | G |
| 100 | 100 | D |
| 200 | 25 | H |
| 200 | 5 | I |
| 200 | 100 | J |

### Comparative Example 7

Formulations Comprising Mixed Oxide Coated Azacitidine, Indomethacin and Lactose Microparticles

The procedure described in Example 2 above was followed to produce suspensions of coated microparticles of azacitidine at final concentrations of 100 mg/mL and 200 mg/mL (referred to hereinafter as ‘Formulation B’ and ‘Formulation E’, respectively), indomethacin (from Comparative Example 5 above) and coated microparticles of lactose (from Comparative Example 6 above) at final concentrations of 100 mg/mL in 2.2 mL of Hyanoate vet, respectively labelled ‘Formulation C’ (indomethacin) and ‘Formulation A’ (lactose).

### Example 3

### Minipig Study I

The objective of this study (which was carried out at Scantox A/S, Denmark) was to assess the local tolerance and pharmacokinetics of azacitidine formulated according to the invention administered by subcutaneous injection to a minipig, as well as local tolerance following administration of azacitidine formulated according to the invention and indomethacin formulated as described herein.

The minipig was selected as the test model because of its well accepted suitability in this type of study and the close resemblance in skin physiology between humans and minipigs. A staggered dose scheme starting with two doses equivalent to ¼ and ½ of the equivalent human clinical dose before going up to the full dose were chosen to reduce the risk for severe local reactions.

The animal had a body weight of 24.9 kg when allocated to the study and was housed in accordance with EU Directive 2010/63/EU of 22 Sep. 2010 on the protection of animals used for scientific purposes. In short, a standard minipig diet was offered twice daily (morning and afternoon) in an amount of approximately 350 g per meal. The amount of diet may be adjusted during the course of the study in order to allow a reasonable growth of the animal. A supply of dehydrated grass (Compact Gras, Hartog B.V., Netherands) was also given daily and the animal had ad libitum access to domestic quality drinking water.

One week prior to start of treatment, the animal was anaesthetised by an intramuscular injection in the neck (1.0 mL/10 kg body weight), and a total of 6 Injection sites (approximately 2×2 cm) were tatooed on back of its neck.

The animal was then anaesthetised again 3 days prior to procedure and an ear vein catheter was implanted to take blood samples during the study. For pain treatment during the study, the animal was given an intramuscular injection in the hind leg of meloxicam 5 mg/mL (0.08 mL/kg) just prior to implantation and once daily for the following two days.

The animal received an intravenous injection of 200 mg ampicillin/mL (0.05 mL/kg). The catheter was flushed with 10 mL of sterile saline and locked using 0.5 mL of TauroLock Hep500 (Taurolidin Citrate with 500IE/mL heparin). A stopper, e.g. Bionector IV access system was applied to the luer.

Between blood sampling occasions, TauroLock™ Hep500 will produce a heparin lock in the catheter.

Single doses of study formulations were given by subcutaneous injection in each of the six marked injection sites as set out in Table 2 below.

TABLE 2

| Injection | | Dose Volume | Dose Amount |
|---|---|---|---|
| Site | Formulation | (mL) | (mg) |
| 1 | A | 1.9 | ca. 200* |
| 2 | B | 0.5 | 50 |
| 3 | B | 1.0 | 100 |
| 4 | B | 0.5 | 50 |
| 5 | D | 0.5 | 50 + 50 |
| 6 | C | 0.5 | 50 |

*Estimated

### Local Tolerance

On Day 1 of the study, the animal was anaesthetized and subcutaneously administered Formulations A and B at injection Sites 1, 2 and 3 in the relevant dose volumes, as set out in Table 2 above. On Day 41 of the study, the animal was anaesthetized and subcutaneously administered Formulations D and C at injection Sites 5 and 6 in the relevant dose volumes, as set out in Table 2 above.

These injections were assessed for local tolerance.

In each case, prior to injection, the relevant sample vials were inverted 3 times just before retracting the sample for each injection to avoid sedimentation of the test material and consequent deviation from the correct dose.

All clinical signs of ill health and any behavioural changes were recorded daily. In addition, dose related observations were performed prior to dosing/in relation to dosing and no earlier than 30 minutes after dosing and any deviation from normal was recorded.

Injection sites were photographed, scored, and recorded at 30 minutes and 2 and 6 hours post dosing and then daily until no score was presented. From Day 10 and onwards, no photographs were taken, and the injection sites were only scored every second day until no score was present or the study had ended.

Injection Sites 5 and 6 was photographed, scored, and recorded at 30 minutes, and 2 and 6 hours post-dosing and daily until Day 48. Thereafter, no photographs were taken, and the injection sites were only scored twice weekly until no score was present or the study had ended.

Particular attention was paid to haemorrhage, erythema, swelling (with indication/measurement of size) and firmness/induration and necrosis, and any other signs of inflammatory or allergic reactions. Parameters were scored according to the following grading system: 0 (not present), 1 (minimal), 2 (slight), 3 (moderate) and 4 (marked).

On Day 2, blood samples were taken from the animal to assess clinical pathology parameters. An additional sampling took place on Day 5. The animal was fasted overnight before blood samples were taken but water was available.

For haematology, at least 2.5 mL K3 EDTA stabilised blood was taken. From this sample, a back-up smear was prepared and stained with May-Grunwald and Giemsa for possible later manual differential leucocyte count. The smears were not analysed and were discarded upon finalisation of the study. For the coagulation tests, 1.8 mL citrate stabilised blood was taken. The parameters, methods and units for the laboratory investigations are presented in Table 3 below.

TABLE 3

| Parameter | Method/Equipment | Units |
|---|---|---|
| Haemoglobin (Hb) | Direct measurement/ABX | mmol/L |
| | Pentra DX120SPS | |
| Red blood cell count (RBC) | Direct measurement/ABX | 10¹²/L |
| | Pentra DX120SPS | |
| Reticulocyte count (RETIC) | Direct measurement/ABX | % and |
| | Pentra DX120SPS | 10¹²/L |
| Haematocrit (HT) | Direct measurement/ABX | L/L |
| | Pentra DX120SPS | |
| Mean cell volume (MCV) | Calculated/ABX Pentra | fL |
| | DX120SPS | |
| Mean cell haemoglobin (MCH) | Calculated/ABX Pentra | fmol |
| | DX120SPS | |
| Mean cell haemoglobin | Calculated/ABX Pentra | mmol/L |
| concentration (MCHC) | DX120SPS | |
| White blood cell count (WBC) | Direct measurement/ABX | 10⁹/L |
| | Pentra DX120SPS | |
| Differential leucocyte count | Direct measurement/ABX | % and |
| (NEUTRO, LYMPHO, | Pentra DX120SPS(**) | 10⁹/L |
| EOS, BASO, MONO) | | |
| Platelet count (Plt) | Direct measurement/ABX | 10⁹/L |
| | Pentra DX120SPS | |
| Activated partial thromboplastin | IL Test ™/ACL ™(*), | sec. |
| time (APTTsyn) | elagic acid as activator | |
| Prothrombin time HS (PT-HS) | IL Test ™/ACL ™(*) | sec. |
| Fibrinogen Clauss (FibC) | IL Test ™/ACL ™(*) | g/L |

(*)Instrumentation Laboratories, Automated Coagulation Laboratory
(**)At the discretion of the technician, individual smears may be manually counted

Approximately 3 mL blood was taken for clinical chemistry in tubes with clotting activator for serum. The parameters, methods and units for the laboratory investigations are presented in Table 4 below.

TABLE 4

| Parameter | Method/Equipment | Units |
|---|---|---|
| Alanine aminotransferase (ALAT) | Cobas 6000 | μkat/L |
| Aspartate aminotransferase (ASAT) | Cobas 6000 | μkat/L |
| Alkaline phosphatase (ALKPH) | Cobas 6000 | μkat/L |
| Bilirubin (total) (TBILI) | Cobas 6000 | μmol/L |
| Gamma-glutamyl transferase (GGT) | Cobas 6000 | μkat/L |
| Cholesterol (CHOL) | Cobas 6000 | mmol/L |
| Triglycerides (TRIG) | Cobas 6000 | mmol/L |
| Carbamide (UREA) | Cobas 6000 | mmol/L |
| Creatinine (CREAT) | Cobas 6000 | μmol/L |
| Glucose (GLUC) | Cobas 6000 | mmol/L |
| Sodium (Na) | Ion selective | mmol/L |
| | electrode/Cobas 6000 | |
| Potassium (K) | Ion selective | mmol/L |
| | electrode/Cobas 6000 | |
| Calcium (Ca) | Cobas 6000 | mmol/L |
| Magnesium (Mg) | Cobas 6000 | mmol/L |
| Inorganic phosphorus (P) | Cobas 6000 | mmol/L |
| Chloride (Cl) | Ion selective | mmol/L |
| | electrode/Cobas 6000 | |
| Protein (total) (PROTEIN) | Cobas 6000 | g/L |
| Albumin (ALB) | Cobas 6000 | g/L |
| Globulin | Calculated | g/L |
| Albumin/Globulin (ALB/G) ratio | Calculated | No unit |

Full-thickness biopsies were taken on Days 3 and 7 from injection Site 1, and on Days 2 and 6 from injection Sites 2 and 3. A single control biopsy was taken outside of the injection site for comparison at the histopathological evaluation. Full-thickness biopsies were taken on Days 43 and 47 from injection Sites 5 and 6.

The animal was anaesthetised prior to biopsy collection and, approximately 30 minutes prior to the first sampling of biopsies, an intramuscular injection of methadone 10 mg/mL (0.02 mL/kg) was given to prevent reactions of pain.

Biopsies were collected using a 8 mm punch and were fixed in phosphate buffered neutral 4% formaldehyde. After fixation, the specimens were trimmed and processed. The specimens were embedded in paraffin and cut at a nominal thickness of approximately 5 μm, stained with haematoxylin and eosin and examined under a light microscope. All pathological findings were entered directly into instem Provantis® (version 9.3.0.0). Histological alterations were graded on a 5-level scale (Minimal, Mild, Moderate, Marked and Severe).

### Pharmacokinetics (PK)

On Day 22 of the study, the animal was anaesthetized and subcutaneously administered Formulation B at Injection Site 4 in the relevant dose volume, as set out in Table 4 above. This injection was assessed for PK parameters.

Prior to injection, the relevant sample vial was inverted 3 times just before retracting the sample as described above for each injection to avoid sedimentation of the test material and consequent deviation from the correct dose.

On the day of dosing, blood samples were taken at the following time points: pre-treatment, and 30 min, 2, 6, 10, 24, 48, 72, 120 and 168 hours post-treatment.

Blood samples of approximately 3 mL was drawn from the jugular vein/bijugular trunk. The blood was sampled into vacutainers containing K2-EDTA as anticoagulant. The vacutainer was placed in ice water until centrifugation (10 min, 1270 G, +4° C.). Each plasma sample was divided into two aliquots of approx. 0.5 mL and transferred to cryotubes and frozen at −18° C. or below within 90 minutes after collection. The first set of samples were sent on dry ice (approximately −70° C.) for analysis (the shipment was sent without thermologger). The second set of samples were stored at −18° C. or below as back-up samples. The back-up samples were shipped a couple of days after receipt of the primary samples.

Azacitidine in plasma was determined by UPLC-MS/MS. Azacitidine was extracted from plasma by protein precipitation using DMF:Acetonitrile (5:95). After injection on a straight phase chromatographic column, the substance was eluted with an acetonitrile and aqueous gradient and detected with MS/MS.

Individual plasma concentration profiles were subjected to non-compartmental pharmacokinetic analysis using the software PKanalix (version 2020).

When the plasma concentration obtained in the pre-dose sample was below LLOQ, the data point was entered as zero. All other concentrations below LLOQ were entered as half the value of LLOQ (½*LLOQ). Consecutive data points below LLOQ after Tₘₐₓ was excluded from modelling and analysis.

The maximum plasma concentration (Cₘₐₓ) and the time when it occurs (Tₘₐₓ) was estimated by visual inspection of the data.

The area under the curve from time zero to the time point of the last quantifiable concentration (AUC₍₀₋ₜ₎) and the area under the curve from time zero to infinity (AUC_{inf}) was calculated according to the linear/log trapezoidal method. If the extrapolated area (AUC_{(% extrapolated)}) constitutes more than 20%, the AUC_{inf} was considered less reliable.

The half-life T_{1/2} was calculated as ln2/1_{z}, where 1_{z} was the elimination rate constant. The half-life was only calculated when at least 3 data points could be included. If the regression line resulted in an Rsq of less than 0.80, the results were not considered reliable.

After collection of the last blood sample/procedure, the animal was no longer part of the study and was terminated.

### Results

At Injection Site 1, after dosing with Formulation A (lactose) initially no reaction was observed. However, at Day 14, a soft swelling was observed (up to 10×15 mm, which was barely perceptible).

The day after dosing with Formulation B at injection Site 2 (azacitidine, 50 mg), a hard swelling (up to 40×20 mm) was observed, which lasted for at least 28 days. Minimal to slight erythema was observed on Days 2 and 3 and again from Day 7. The day after dosing with Formulation B at injection Site 4 (also azacitidine, 50 mg), a soft swelling (up to 10×10 mm, which was barely perceptible), which lasted until 11 days after dosing (Days 23 to 33). Minimal erythema was observed from Days 23 to 28.

However, the day after dosing with Formulation B at injection Site 3 (azacitidine, 100 mg), a hard, well-defined swelling at the injection site (up to 55×30 mm, which lasted at least 28 days. Slight erythema was observed on Days 2 and 3 and again from Day 7.

Conversely, the day after dosing with Formulation D at Injection Site 5 (azacitidine 50 mg plus indomethacin 50 mg), no injection site reaction whatsoever was observed.

In terms of PK parameters, the single subcutaneous administration of 50 mg of coated azacitidine (Injection Site 4) showed a systemic exposure with prolonged release profile as shown in FIG. 3. The duration was 120 h and 47% of the exposure was observed within the first 12 h post administration.

The histopathology results demonstrated the following: Injection Site 2: moderate inflammation and moderate necrosis at Day 3 and mild inflammation and moderate necrosis at Day 7.

Injection Site 5 moderate inflammation and mild necrosis at Day 45 (3 days after injection) and minimal inflammation and minimal necrosis at Day 49 (7 days after injection).

It was concluded that subcutaneous administration of Formulation D (combination of mixed oxide coated azacitidine and mixed oxide coated indomethacin) caused less skin reaction than subcutaneous administration of Formulation B (mixed oxide coated azacitidine alone).

### Example 4

### Mini Pig Study II

A similar study to that described in Example 3 above was conducted on five minipigs. On the day of arrival, the animals were given a final number, using a randomisation scheme. The animals received a chip with a unique numerical code.

Approximately one week prior to start of treatment, two injection sites were marked on necks of minipigs in the same manner as described in Example 3 above

The animals' treatment schedule was as set out in Table 5 below, which refers to specific formulations prepared in accordance with the relevant examples and comparative above. Vidaza® (Mylan), the commercial injectable formulation of azacitidine with a concentration of 25 mg/mL can be considered to provide an equivalent dose of ‘uncoated’ azacitidine.

TABLE 5

| | | | Dose | | |
|---|---|---|---|---|---|
| Animal/ | Injection | Formu- | Volume | Dose(s) (mg) | |
| Group | Site | lation | (mL) | Azacitidine | Indomethacin |
| 1 | 1 | B | 0.5 | 50 | 0 |
| | 2 | F | 0.5 | 50 | 12.5 |
| 2 | 1 | G | 0.5 | 50 | 2.5 |
| | 2 | D | 0.5 | 50 | 50 |
| 3 | 1 | E | 0.25 | 50 | 0 |
| | 2 | H | 0.25 | 50 | 6.25 |
| 4 | 1 | I | 0.25 | 50 | 1.25 |
| | 2 | Vidaza | 2 | 50 | 0 |
| 5 | 1 | Vidaza | 2 | 50 | 0 |
| | 2 | J | 0.25 | 50 | 25 |

Essentially the same treatment protocol as that described in Example 3 above was followed. For each animal subcutaneous injections of relevant formulations were administered at the above dose volumes on Day 1 (Injection Site 1 on each animal) and Day 8 (Injection Site 2 on each animal).

Observations were carried out in essentially the same manner as described in Example 3 above, with necropsy on Day 29.

The size of the inflammatory swelling in mm³ is presented in FIG. 4 for:
Animal 1/Injection Site 1 (50 mg azacitidine, lower concentration of particles; diamonds);
Animal 3/Injection Site 1 (50 mg azacitidine, higher concentration of particles; triangles);
Animal 2/Injection Site 2 (50 mg azacitidine+lower concentration of particles 50 mg indomethacin; crosses); and
Animal 5/Injection Site 2 (50 mg azacitidine+25 mg indomethacin; squares).

FIG. 4 clearly shows the huge impact of administration of azacitidine with indomethacin.

1. An injectable pharmaceutical or veterinary formulation comprising:
(a) a biologically-active agent in admixture with a pharmaceutically- or veterinarily-acceptable extended-release component;
(b) an antiinflammatory agent; and
(c) a pharmaceutically- or veterinarily-acceptable injectable carrier.

2. A formulation as claimed in claim 1, which is suitable for subcutaneous or intramuscular injection to form a depot.

3. A formulation as claimed in claim 1 or claim 2 wherein an extended-release component is also applied to the antiinflammatory agent within the formulation of the invention.

4. A formulation as claimed in claim 3, wherein the extended-release components provide for an extended release of said biologically active agent and said antiinflammatory agent at essentially the same rate, and/or over essentially the same period of time.

5. A formulation as claimed in any one the preceding claims which comprises:
(a) a plurality of particles having a weight-, number-, or volume-based mean diameter that is between about 10 nm and about 700 μm, which particles comprise solid cores comprising a biologically-active agent coated with a coating comprising at least one coating material applied by way of a gas phase deposition technique;
(b) which particles are suspended in a carrier system comprising a pharmaceutically-acceptable or veterinarily-acceptable vehicle; and
(c) which formulation further includes an antiinflammatory agent.

6. A formulation as claimed in claim 5, wherein the coating material comprises one or more metal oxide.

7. A formulation as claimed in claim 6, wherein the coating material comprises zinc oxide.

8. A formulation as claimed in any one of claims 5 to 7, wherein the coating material comprises at least one separate mixture of zinc oxide and one or more other metal and/or metalloid oxides in a atomic ratio of between about 1:10 (e.g. about 1:6 or about 1:1) up to and including about 10:1 (e.g. about 6:1).

9. A formulation as claimed in claim 8, wherein the one or more other metal and/or metalloid oxides are selected from aluminium oxide and/or silicon dioxide.

10. A formulation as claimed in any one of claims 5 to 9, wherein the cores are coated with one or more discrete layers surrounding said cores.

11. A formulation as claimed in claim 10, wherein more than one discrete layers of coating materials are applied to the core sequentially.

12. A formulation as claimed in claim 11, wherein between 2 and 10 discrete layers of the coating materials are applied.

13. A formulation as claimed in any one of claims 5 to 12, wherein the weight-, number-, or volume-based mean diameter of the particles is between about 1 μm and about 50 μm.

14. A formulation as claimed in any one of claims 5 to 13, wherein the total thickness of the coating is between about 0.5 nm and about 2 μm.

15. A formulation as claimed in any one of claims 5 to 14, wherein the gas phase deposition technique is atomic layer deposition.

16. A formulation as claimed in any one of the preceding claims in the form of a sterile injectable and/or infusible dosage form.

17. A formulation as claimed in claim 16 in a form that is administrable via a surgical administration apparatus that forms a depot formulation.

18. A formulation as claimed in any one of the preceding claims wherein the biologically active agent produces an inflammatory response when administered to a patient, or may be expected to produce such a response.

19. A formulation as claimed in claim 18, wherein the biologically active agent is selected from the group: antineoplastic agents, topoisomerase inhibitors, immunomodulators, immunostimulants, immunosuppressants, chemotherapeutics, growth factors, vasodilators, radiopharmaceuticals and combinations thereof.

20. A formulation as claimed in claim 18 or claim 19, wherein the biologically active agent is a cytokine, a protein, a vaccine or a peptide.

21. A formulation as claimed in any one of claims 18 to 20, wherein the biologically active agent is selected from the group: daratumumab, isatuximab, actinomycin, azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, daunorubicin, decitabine, docetaxel, doxifluridine, doxorubicin, epirubicin, epothilone, estramustin, etoposide, everolimus, fludarabine, fluorouracil, guadecitabine, gemcitabine, hydroxyurea, idarubicin, Ifosfamide, irinotecan, ixazomib, karfilzomib, lomustin, mechlorethamine, melphalan, mercaptopurine, mesna, methotrexate, mitotan, mitoxantrone, nelarabin, oxaliplatin, paclitaxel, panobinostat, pemetrexed, pixantron, procarbazine, tegafur, temozolomide, teniposide, tioguanine, tiotepa, topotecan, trabektedin, valrubicin, venetoclax, vinblastine, vincristine, vindesine, vinflunine, vinorelbine, bendamustine, bleomycin, carboplatin, chlorambucil, cisplatin, cyclophosphamide, cyclosporin, cytarabine, dacarbazine, dactinomycin, daunorubicin, docetaxel, doxorubicin, epirubicin, etoposide, everolimus, fluorouracil, gemcitabine, ifosfamide, Irinotecan, mercaptopurine, mesna, methotrexate, midazolam, mitomycin, oxaliplatin, paclitaxel, procarbazine, temsirolimus, thioguanine, vinblastine, vincristine, vinorelbine, as thalidomide, pomalidomide, lenalidomide, apremilast, pharmaceutically acceptable salts of any of these active ingredients, and combinations thereof.

22. A formulation as claimed in claim 21, wherein the biologically active agent is selected from the group azacitidine and lenalidomide.

23. A formulation as claimed in any one of the preceding claims, wherein the antiinflammatory agent is selected from the group: a butylpyrazolidine, an acetic acid derivative or a related substance, an oxicam, a propionic acid derivative, a fenamate, a coxib, a non-steroidal antiinflammatory agent, a corticosteroid, a quinoline, a gold preparation, an antihistamine, and combinations thereof.

24. A formulation as claimed in any one of the preceding claims, wherein the antiinflammatory agent is selected from the group: phenylbutazone, mofebutazone, oxyphenbutazone, clofezone, kebuzone, indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, piroxicam, tenoxicam, droxicam, lornoxicam, meloxicam, ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, vedaprofen, carprofen, tepoxalin, mefenamic acid, tolfenamic acid, flufenamic acid, meclofenamic acid, flunixin, celecoxib, rofecoxib, valdecoxib, parecoxib, etoricoxib, lumiracoxib, firocoxib, robenacoxib, mavacoxib, cimicoxib, nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, pentosan polysulfate, aminopropionitrile, 11-dehydrocorticosterone, 11-deoxycorticosterone, 11-deoxycortisol, 11-ketoprogesterone, 11β-hydroxypregnenolone, 11β-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone (prebediolone), aldosterone, corticosterone (17-deoxycortisol), cortisol (hydrocortisone), cortisone, pregnenolone, progesterone, flugestone (flurogestone), fluorometholone, medrysone (hydroxymethylprogesterone), prebediolone acetate (21-acetoxypregnenolone), chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol (halobetasol), amcinonide, budesonide, ciclesonide, deflazacort, desonide, formocortal fluclorolone acetonide (flucloronide), fludroxycortide (flurandrenolone, flurandrenolide), flunisolide, fluocinolone acetonide, fluocinonide, halcinonide, triamcinolone acetonide, oxycinchophen, sodium aurothiomalate, sodium aurothiosulfate, auranofin, aurothioglucose, aurotioprol, penicillamine, bucillamine, akrivastin, alimemazin, antazolin, astemizol, azatadin, azelastin, bamipin, bilastin, bromdifenhydramin, bromfeniramin, buklizin, cetirizin, cinnarizine, cyklizin, cyproheptadine, deptropine, desloratadin, dexbromfeniramin, dexklorfeniramin, difenylpyralin, dimenhydrinat, dimetinden, doxylamin, ebastin, epinastin, fenindamin, feniramin, fexofenadin, histapyrrodin, hydroxietylprometazin, isotipendyl, karbinoxamin, ketotifen, kifenadin, klemastin, klorcyklizin, klorfenamin, klorfenoxamin, kloropyramin, levocetirizin, loratadin, mebhydrolin, mekitazin, meklozin, mepyramin, metapyrilen, metdilazin, mizolastin, oxatomide, oxomemazine, pimetixen, prometazin, pyrrobutamin, rupatadin, sekifenadin, talastin, tenalidin, terfenadin, tiazinam, tietylperazin, tonzylamin, trimetobenzamid, tripelennamin, triprolidine, tritokvalin, pharmaceutically acceptable salts of any of these active ingredients, and combinations thereof.

25. A formulation as claimed in any one of claims 5 to 24, wherein the antiinflammatory agent is formulated along with the biologically-active agent within the solid cores that form part of a formulation of the invention.

26. A formulation as claimed in any one of the preceding claims, wherein the antiinflammatory agent is dissolved, and/or suspended, within the carrier system.

27. A formulation as claimed in claim 26, wherein the antiinflammatory agent is provided in the form of additional particles having a weight-, number-, or volume-based mean diameter that is between about 10 nm and about 700 μm, and comprising cores comprising said antiinflammatory agent, which cores are coated, at least in part, by one or more coating materials, which allows for the release of the antiinflammatory agent over the same, or over a different timescale, to the biologically-active agent.

28. A formulation as claimed in claim 27, wherein the coating is as defined in any one of claims 5 to 12.

29. A process for the preparation of a formulation as defined in any one of claims 5 to 28, wherein the coated particles are made by applying the layer(s) of coating material to the cores, and/or previously-coated cores, the gas phase deposition technique.

30. A process as claimed in claim 29, wherein:
(i) solid cores are coated with a first discrete layer of coating material;
(ii) the coated cores from step (i) are then subjected to a deagglomeration process step;
(iii) the deagglomerated coated cores from step (ii) are then coated with a second discrete layer of coating material;
(iv) repeating steps (ii) and (iii) to obtain the required number of discrete layers.

31. A process as claimed in claim 30, wherein the deagglomeration step that takes place between applications of coatings comprises sieving.

32. A process as claimed in claim 31, wherein the sieving comprises vibrational sieving.

33. A process as claimed in claim 32, wherein the vibrational sieving comprises controlling a vibration probe coupled to the sieve.

34. A process as claimed in claim 31, wherein the sieving comprises sonic sifting.

35. A process for the preparation of a formulation as defined in any one of claims 5 to 28 wherein the coated particles are mixed with the carrier system after coating.

36. An injectable and/or infusible dosage form comprising a formulation as defined in any one of claims 1 to 28 contained within a reservoir that is connected to, and/or is associated with, an injection or infusion means.

37. A dosage form as claimed in claim 36, which is a surgical administration apparatus that forms a depot formulation.

38. A dosage form as claimed in claim 36 or claim 37, wherein coated particles as defined in any one of claims 1 to 28, and the carrier system, are housed separately, and in which admixing occurs prior to and/or during injection or infusion.

39. A method of treatment of a disease or a condition a patient, which comprises administration of a formulation as defined in any one or claims 1 to 28 or a dosage form as claimed in any one of claims 36 to 38, wherein the biologically active agent that is employed in said formulation or said dosage form is suitable for use in said disease or condition.

40. A method of treatment of a disease or a condition a patient, which comprises administration of:
(A) a formulation as defined in any one or claims 1 to 28, or a formulation that is, in all other respects, a formulation as defined in any one or claims 1 to 28, provided that it does not comprise an antiinflammatory agent, or a dosage form as defined in any one of claims 36 to 38 containing any such formulation, and wherein the biologically active agent that is employed in said formulation or said dosage form is suitable for use in said disease or condition; and
(B) a pharmaceutical formulation comprising an antiinflammatory agent in admixture with one or more a pharmaceutically- or veterinarily-acceptable excipients, such as a carrier system as hereinbefore defined or in accordance with the invention, in which method, Components (A) and (B) are each provided in a form that is suitable for administration in conjunction with each other.

41. A method of treatment of a disease or a condition a patient, which method comprises bringing Component (A), as defined in claim 40, into association with a Component (B), as defined in claim 40 above, thus rendering the two components suitable for administration in conjunction with each other.

42. A method of treatment of a disease or a condition a patient, which comprises:
(i) administration of Component (A) as defined in claim 40 to a patient having the a relevant disease or condition; and
(ii) if that patient exhibits an inflammatory response after such administration, administering:
a. a formulation as defined in any one or claims 1 to 28,
b. a dosage form as defined in any one of claims 36 to 38, or
c. Component (B) as defined in claim 40,
to said patient.

43. A method of treatment as claimed in any one of claims 39 to 42 wherein the disease of condition is cancer, and the biologically active agent that is employed in the formulation is an injectable anticancer drug.

44. An injectable anticancer drug for use in a method of treatment as claimed in any one of claims 39 to 42.

45. The use of an injectable anticancer drug for the manufacture of a medicament for a method of treatment as claimed in any one of claims 39 to 42.

46. A method as claimed in claim 43, a compound for use as claimed in claim 44, or a use as claimed in claim 45, wherein the biologically active agent is azacitidine or lenalidomide.

47. A kit of parts comprising:
(I) one of Components (A) and (B) as defined in claim 40; together with
(II) Instructions to use that component in conjunction with the other of the two components.

48. A kit of parts comprising Components (A) and (B) as defined in claim 40, packaged and presented together as separate components of a combination pack, for use in conjunction with each other.
